SEQUENCE LISTING (5 sequences)

SEQ ID NO: 1 - DNA, 6385 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 6124 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttgccaccatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 7102 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttgccaccatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 4 - DNA, 8307 bp, Artificial Sequence
  Description of Artificial Sequence Synthetic polynucleotide
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattcttgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttgccaccatgagctccggcaacatgaaagataagcacagaagctacaagagaaagggctctccagagaggggcgagcgcaagcggcactggcagacaacccaccacagatctagatccagaagccccattagacattctggagagcggggaagcggcagctatcaccaggagcaccctatcagccacctgagcagctgcaccgcctctaagaccagcgaccaagtgatgaagacaagagaatctacaagcggcaagaaagacaacagaacaaacccctacacagtgttcagccagcaccgggccagcaatcctgaggctcctggctggtgcggcttctactggcacagcaccagaatcgccagagatggcaccaacagcatcttcaacgagatgaagcagcaatttcagcagctgcagatcgacaacaagatcggctgggataacacccgggaactgctgtttaaccagaaaaagaccctggaccagaagtacagaaatatgttctggcatttcagaaacaacagcgactgtgaacggtgcaactactgggacgacgtgtaccggagacacctggccaacgtctcctcccagaccgaggccgatgaaatcaccgacgaggaaatgctgagcgccgccgagagcatggaagctgacgcctctaattgagcgtacagcggctcccgggagttctagggatctgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataaggatccaccggaggccaccatggccttcaacccccccgtgatccgggcctttagccagcccgcttttacatacgtgttcaagttcccctacccccagtggaaggaaaaggaatggctgctgcacgccctgctggcccacggcaccgagcagtccatgatccagctgcggaactgcgccccacaccccgatgaagacatcattagagacgaccttctgatcagcctggaagatagacacttcggcgccgttctgtgcaaagccgtgtacatggccaccaccaccctcatgagccacaagcagagaaatatgttcccccggtgcgacatcatcgtccagagcgagctgggagaaaagaatctccattgtcacatcattgtgggcggcgagggcctgtccaagagaaacgccaagtcctcttgcgcccagttctacggcctgatcctggctgagatcatccagaggtgtaaaagcctgctggccaccagaccattcgaacctgaggaagccgacatcttccacaccctgaagaaggccgagcgggaggcctggggaggcgtgacaggcggcaacatgcaaatcctgcaatacagagatagaagaggcgacctgcatgcacaaacagtggatcctctgagattcttcaagaattacctgctgcctaagaacagatgcatcagctcttacagcaagcccgatgtgtgcacctctcctgacaactggttcatcctggccgagaagacctacagccacacactgatcaacggcctgcccctgcctgaacactaccggaagaactaccacgccaccctggacaacgaagtgatccccggccctcagaccatggcttatggaggccggggaccatgggagcacctgccagaggactttaccctgcacgagaacggatactgcacagactgtggcggatatctgcctcacagcgccgacaacagcatgtacaccgacagagccagcgagacatctaccggcgacatcacacctagcgacctgggcgatagcgacggcgaggacaccgagcctgagacaagccaggtggactactgtcctcctaagaaaagacgcctgaccgcccctgctagccctccaaacagccctgccagctctgtgtccaccatcacattctttaatacctggcacgcccagcctagagatgaggatgagctgagagagtacgagcggcaggccagccttctgcagaaaaagcgggagagcagaaagcgcggcgaagaggaaacactggctgacaacagctcccaggagcaggagcctcagcctgatcctacccagtggggcgaaagactgggcttcatcagctctggcacgcccaaccagccccctatcgtgctgcactgcttcgaagatctgagaccttctgacgaggacgaaggtgaatacatcggcgaaaaaagacagtagctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 5 - DNA, 13838 bp, Artificial Sequence
  Description of Artificial Sequence Synthetic polynucleotide
ggtacccaactccatgcttaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactaggagacactttcaataaaggcaaatgtttttatttgtacactctcgggtgattatttaccccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggtgcatgcccaggctttgagttgcactcgcaccgtagtggcatcagaaggtgaccgtgcccggtctgggcgttaggatacagcgcctgcatgaaagccttgatctgcttaaaagccacctgagcctttgcgccttcagagaagaacatgccgcaagacttgccggaaaactgattggccggacaggccgcgtcatgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgctcccgtgtagacacttaagctcgccttcgatctcagcgcagcggtgcagccacaacgcgcagcccgtgggctcgtggtgcttgtaggttacctctgcaaacgactgcaggtacgcctgcaggaatcgccccatcatcgtcacaaaggtcttgttgctggtgaaggtcagctgcaacccgcggtgctcctcgtttagccaggtcttgcatacggccgccagagcttccacttggtcaggcagtagcttgaagtttgcctttagatcgttatccacgtggtacttgtccatcaacgcgcgcgcagcctccatgcccttctcccacgcagacacgatcggcaggctcagcgggtttatcaccgtgctttcactttccgcttcactggactcttccttttcctcttgcgtccgcataccccgcgccactgggtcgtcttcattcagccgccgcaccgtgcgcttacctcccttgccgtgcttgattagcaccggtgggttgctgaaacccaccatttgtagcgccacatcttctctttcttcctcgctgtccacgatcacctctggggatggcgggcgctcgggcttgggagaggggcgcttctttttctttttggacgcaatggccaaatccgccgtcgaggtcgatggccgcgggctgggtgtgcgcggcaccagcgcatcttgtgacgagtcttcttcgtcctcggactcgagacgccgcctcagccgcttttttgggggcgcgcggggaggcggcggcgacggcgacggggacgacacgtcctccatggttggtggacgtcgcgccgcaccgcgtccgcgctcgggggtggtttcgcgctgctcctcttcccgactggccatttccttctcctataggcagaaaaagatcatggagtcagtcgagaaggaggacagcctaaccgccccctttgagttcgccaccaccgcctccaccgatgccgccaacgcgcctaccaccttccccgtcgaggcacccccgcttgaggaggaggaagtgattatcgagcaggacccaggttttgtaagcgaagacgacgaggatcgctcagtaccaacagaggataaaaagcaagaccaggacgacgcagaggcaaacgaggaacaagtcgggcggggggaccaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctgttctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcgacgaagtgccaaaaatctttgagggtcttggacgcgacgagaaacgcgcggcaaacgctctgcaacaagaaaacagcgaaaatgaaagtcactgtggagtgctggtggaacttgagggtgacaacgcgcgcctagccgtgctgaaacgcagcatcgaggtcacccactttgcctacccggcacttaacctaccccccaaggttatgagcacagtcatgagcgagctgatcgtgcgccgtgcacgacccctggagagggatgcaaacttgcaagaacaaaccgaggagggcctacccgcagttggcgatgagcagctggcgcgctggcttgagacgcgcgagcctgccgacttggaggagcgacgcaagctaatgatggccgcagtgcttgttaccgtggagcttgagtgcatgcagcggttctttgctgacccggagatgcagcgcaagctagaggaaacgttgcactacacctttcgccagggctacgtgcgccaggcctgcaaaatttccaacgtggagctctgcaacctggtctcctaccttggaattttgcacgaaaaccgcctcgggcaaaacgtgcttcattccacgctcaagggcgaggcgcgccgcgactacgtccgcgactgcgtttacttatttctgtgctacacctggcaaacggccatgggcgtgtggcagcaatgcctggaggagcgcaacctaaaggagctgcagaagctgctaaagcaaaacttgaaggacctatggacggccttcaacgagcgctccgtggccgcgcacctggcggacattatcttccccgaacgcctgcttaaaaccctgcaacagggtctgccagacttcaccagtcaaagcatgttgcaaaactttaggaactttatcctagagcgttcaggaattctgcccgccacctgctgtgcgcttcctagcgactttgtgcccattaagtaccgtgaatgccctccgccgctttggggtcactgctaccttctgcagctagccaactaccttgcctaccactccgacatcatggaagacgtgagcggtgacggcctactggagtgtcactgtcgctgcaacctatgcaccccgcaccgctccctggtctgcaattcgcaactgcttagcgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacatccttggccaattgcaagccatcaacaaagcccgccaagagtttctgctacgaaagggacggggggtttacctggacccccagtccggcgaggagctcaacccaatccccccgccgccgcagccctatcagcagccgcgggcccttgcttcccaggatggcacccaaaaagaagctgcagctgccgccgccgccacccacggacgaggaggaatactgggacagtcaggcagaggaggttttggacgaggaggaggagatgatggaagactgggacagcctagacgaagcttccgaggccgaagaggtgtcagacgaaacaccgtcaccctcggtcgcattcccctcgccggcgccccagaaattggcaaccgttcccagcatcgctacaacctccgctcctcaggcgccgccggcactgcctgttcgccgacccaaccgtagatgggacaccactggaaccagggccggtaagtctaagcagccgccgccgttagcccaagagcaacaacagcgccaaggctaccgctcgtggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccctactgcaccggcggcagcggcagcggcagcaacagcagcggtcacacagaagcaaaggcgaccggatagcaagactctgacaaagcccaagaaatccacagcggcggcagcagcaggaggaggagcgctgcgtctggcgcccaacgaacccgtatcgacccgcgagcttagaaataggatttttcccactctgtatgctatatttcaacaaagcaggggccaagaacaagagctgaaaataaaaaacaggtctctgcgctccctcacccgcagctgcctgtatcacaaaagcgaagatcagcttcggcgcacgctggaagacgcggaggctctcttcagcaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcctcgaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcgttttagggcggagtaacttgcatgtattgggaattgtagtttttttaaaatgggaagtgacgtatcgtgggaaaacggaagtgaagatttgaggaagttgtgggttttttggctttcgtttctgggcgtaggttcgcgtgcggttttctgggtgttttttgtggactttaaccgttacgtcattttttagtcctatatatactcgctctgtacttggccctttttacactgtgactgattgagctggtgccgtgtcgagtggtgttttttaataggtttttttactggtaaggctgactgttatggctgccgctgtggaagcgctgtatgttgttctggagcgggagggtgctattttgcctaggcaggagggtttttcaggtgtttatgtgtttttctctcctattaattttgttatacctcctatgggggctgtaatgttgtctctacgcctgcgggtatgtattcccccgggctatttcggtcgctttttagcactgaccgatgttaaccaacctgatgtgtttaccgagtcttacattatgactccggacatgaccgaggaactgtcggtggtgctttttaatcacggtgaccagtttttttacggtcacgccggcatggccgtagtccgtcttatgcttataagggttgtttttcctgttgtaagacaggcttctaatgtttaaatgtttttttttttgttattttattttgtgtttaatgcaggaacccgcagacatgtttgagagaaaaatggtgtctttttctgtggtggttccggaacttacctgcctttatctgcatgagcatgactacgatgtgcttgcttttttgcgcgaggctttgcctgattttttgagcagcaccttgcattttatatcgccgcccatgcaacaagcttacataggggctacgctggttagcatagctccgagtatgcgtgtcataatcagtgtgggttcttttgtcatggttcctggcggggaagtggccgcgctggtccgtgcagacctgcacgattatgttcagctggccctgcgaagggacctacgggatcgcggtatttttgttaatgttccgcttttgaatcttatacaggtctgtgaggaacctgaatttttgcaatcatgattcgctgcttgaggctgaaggtggagggcgctctggagcagatttttacaatggccggacttaatattcgggatttgcttagagacatattgataaggtggcgagatgaaaattatttgggcatggttgaaggtgctggaatgtttatagaggagattcaccctgaagggtttagcctttacgtccacttggacgtgagggcagtttgccttttggaagccattgtgcaacatcttacaaatgccattatctgttctttggctgtagagtttgaccacgccaccggaggggagcgcgttcacttaatagatcttcattttgaggttttggataatcttttggaataaaaaaaaaaaaacatggttcttccagctcttcccgctcctcccgtgtgtgactcgcagaacgaatgtgtaggttggctgggtgtggcttattctgcggtggtggatgttatcagggcagcggcgcatgaaggagtttacatagaacccgaagccagggggcgcctggatgctttgagagagtggatatactacaactactacacagagcgagctaagcgacgagaccggagacgcagatctgtttgtcacgcccgcacctggttttgcttcaggaaatatgactacgtccggcgttccatttggcatgacactacgaccaacacgatctcggttgtctcggcgcactccgtacagtagggatcgcctacctccttttgagacagagacccgcgctaccatactggaggatcatccgctgctgcccgaatgtaacactttgacaatgcacaacgtgagttacgtgcgaggtcttccctgcagtgtgggatttacgctgattcaggaatgggttgttccctgggatatggttctgacgcgggaggagcttgtaatcctgaggaagtgtatgcacgtgtgcctgtgttgtgccaacattgatatcatgacgagcatgatgatccatggttacgagtcctgggctctccactgtcattgttccagtcccggttccctgcagtgcatagccggcgggcaggttttggccagctggtttaggatggtggtggatggcgccatgtttaatcagaggtttatatggtaccgggaggtggtgaattacaacatgccaaaagaggtaatgtttatgtccagcgtgtttatgaggggtcgccacttaatctacctgcgcttgtggtatgatggccacgtgggttctgtggtccccgccatgagctttggatacagcgccttgcactgtgggattttgaacaatattgtggtgctgtgctgcagttactgtgctgatttaagtgagatcagggtgcgctgctgtgcccggaggacaaggcgtctcatgctgcgggcggtgcgaatcatcgctgaggagaccactgccatgttgtattcctgcaggacggagcggcggcggcagcagtttattcgcgcgctgctgcagcaccaccgccctatcctgatgcacgattatgactctacccccatgtaggcgtggacttccccttcgccgcccgttgagcaaccgcaagttggacagcagcctgtggctcagcagctggacagcgacatgaacttaagcgagctgcccggggagtttattaatatcactgatgagcgtttggctcgacaggaaaccgtgtggaatataacacctaagaatatgtctgttacccatgatatgatgctttttaaggccagccggggagaaaggactgtgtactctgtgtgttgggagggaggtggcaggttgaatactagggttctgtgagtttgattaaggtacggtgatcaatataagctatgtggtggtggggctatactactgaatgaaaaatgacttgaaattttctgcaattgaaaaataaacacgttgaaacataacatgcaacaggttcacgattctttattcctgggcaatgtaggagaaggtgtaagagttggtagcaaaagtttcagtggtgtattttccactttcccaggaccatgtaaaagacatagagtaagtgcttacctcgctagtttctgtggattcactagaatcgatgtaggatgttgcccctcctgacgcggtaggagaaggggagggtgccctgcatgtctgccgctgctcttgctcttgccgctgctgaggaggggggcgcatctgccgcagcaccggatgcatctgggaaaagcaaaaaaggggctcgtccctgtttccggaggaatttgcaagcggggtcttgcatgacggggaggcaaacccccgttcgccgcagtccggccggcccgagactcgaaccgggggtcctgcgactcaacccttggaaaataaccctccggctacagggagcgagccacttaatgctttcgctttccagcctaaccgcttacgccgcgcgcggccagtggccaaaaaagctagcgcagcagccgccgcgcctggaaggaagccaaaaggagcgctcccccgttgtctgacgtcgcacacctgggttcgacacgcgggcggtaaccgcatggatcacggcggacggccggatccggggttcgaaccccggtcgtccgccatgatacccttgcgaatttatccaccagaccacggaagagtgcccgcttacaggctctccttttgcacggtctagagcgtcaacgactgcgcacgcctcaccggccagagcgtcccgaccatggagcactttttgccgctgcgcaacatctggaaccgcgtccgcgactttccgcgcgcctccaccaccgccgccggcatcacctggatgtccaggtacatctacggattacgtcgacgtttaaaccatatgatcagctcactcaaaggcggtaatacggttatccacagaatcaggggataacgcaggaaagaacatgtgagcaaaaggccagcaaaaggccaggaaccgtaaaaaggccgcgttgctggcgttgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataagaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatcccctttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgatggatcc

(19) United States
(12) Patent Application Publication
(10) Pub. No.: US 2022/0259572 A1
(43) Pub. Date: Aug. 18, 2022

(54) ADENO-ASSOCIATED VIRUS (AAV) PRODUCTION

(71) Applicant: LONZA HOUSTON, INC., Houston, TX (US)

(72) Inventors: Bingnan GU, Houston, TX (US); Jennifer WANG, Houston, TX (US)

(21) Appl. No.: 17/669,033

(22) Filed: Feb. 10, 2022

Related U.S. Application Data
(60) Provisional application No. 63/148,350, filed on Feb. 11, 2021.

(51) Int. Cl. C12N 7/00; C12N 15/85

(52) U.S. Cl. CPC C12N 7/00; C12N 2750/14143; C12N 2750/14152; C12N 15/85

(57) ABSTRACT

The disclosure relates to methods for producing an adeno-associated virus (AAV) in an E1 complementary producer cell.

### SEQUENCE LISTING

The instant application contains a Sequence Listing which has been submitted electronically in ASCII format and is hereby incorporated by reference in its entirety. Said ASCII copy, created on Feb. 7, 2022, is named 0132-0156US1_SL.txt and is 55,555 bytes in size.

### FIELD OF THE INVENTION

The present disclosure relates to methods for producing an adeno-associated virus (AAV) in an E1 complementary producer cell.

### BACKGROUND

Adeno-associated virus (AAV) is a non-enveloped DNA virus. As a leading viral vector for in vivo gene therapy, AAV is non-pathogenic and can effectively infect a wide range of human tissues. AAV genome includes a 4.7 kb single-stranded DNA that encodes two major genes: the Rep gene encodes four isoforms (Rep78, Rep68, Rep52 and Rep40) that support AAV DNA replication, packaging, integration, and transcription; the Cap gene synthesizes three VP proteins (VP1, VP2, and VP3) for the viral protein shell or capsid construction.

Wild-type AAV cannot replicate without helper viruses, such as adenovirus (Ad) and herpes simplex virus (HSV). Adenoviral helper genes have been identified as necessary and sufficient for AAV replication, including E1A, E1B, E2A, E4, and VA. The adenovirus type 5 (Ad5) 5′ 4.3 kb genomic DNA has been integrated into chromosome 19 of human embryonic kidney 293 (HEK293) cells. This Ad5 genomic fragment expresses all E1A (e.g., 289R, 243R, 171R) and E1B (e.g., 19K and 55K) isoforms. Further cell lines having E1A and E1B integration have been developed, including, e.g., PER.C6 cells and CAP® cells. See, e.g., Fallaux et al., Human Gene Ther 9(13):1909-1917 (1998) and Schiedner et al., Human Gene Ther 11(15):2105-2116 (2000). Accordingly, these cells already express E1A and E1B and therefore can support recombinant AAV (rAAV) production by the “triple plasmid transfection” approach, i.e., transfecting helper plasmid (pHelper) that encodes only E2A, E4orf6, and VA, together with plasmids that encode the Rep and Cap genes (pRepCap) and the gene of interest (pAAV-GOI). Such cells, which have genome-integrated E1A and E1B genes and natively express E1A and E1B proteins, are known as “E1 complementary producer cells.”

Currently, over 70% of the AAV-based gene therapy pipelines in the world rely on the manufacturing platform via transient transfection in HEK293 cells with the standard triple plasmid transfection method. Productivity of the HEK293 and other E1 complementary producer cell manufacturing platforms may not be able to meet the growing demand for AAVs, e.g., for clinical development and commercialization. However, current approaches for increasing AAV productivity in E1 complementary producer cells such as HEK293 cells, e.g., high producer cell clone screening and isolation, transfection protocol optimization, and manufacturing process development, are costly and time-consuming.

### SUMMARY OF THE INVENTION

In some embodiments, the disclosure provides a method of producing an adeno-associated virus (AAV) in an E1 complementary producer cell, comprising: (a) transfecting the E1 complementary producer cell with one or more vectors comprising: (i) an E1A adenovirus helper gene; (ii) an adenovirus helper gene selected from E2A, E4, or both; (iii) a viral-associated, non-coding RNA (VA RNA); and (iv) an AAV gene selected from Rep, Cap, or both; (b) culturing the transfected E1 complementary producer cell under conditions suitable for producing the AAV; and (c) purifying the AAV from the cultured E1 complementary producer cell, thereby obtaining the AAV.

In additional embodiments, the disclosure provides a method of producing an adeno-associated virus (AAV) in an E1 complementary producer cell, comprising: (a) transfecting the E1 complementary producer cell with: (i) a first vector comprising E1A operably linked to a first promoter; (ii) a second vector comprising E2A, E4, and a viral-associated, non-coding RNA (VA RNA), all operably linked to a second promoter; and (iii) a third vector comprising Rep and Cap, both operably linked to a third promoter, wherein a transfection ratio of (i) to each of (ii) and (iii) is about 1:1 to about 3:1; (b) culturing the transfected E1 complementary producer cell under conditions suitable for producing the AAV; and (c) purifying the AAV from the cultured E1 complementary producer cell, thereby obtaining the AAV. Suitably, one or more of the first, second, or third vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI.

In further embodiments, the disclosure provides a method of producing an adeno-associated virus (AAV) in an E1 complementary producer cell, comprising: (a) transfecting the E1 complementary producer cell with: (i) a first vector comprising E1A operably linked to a first promoter and an enhancer; (ii) a second vector comprising E2A, E4, and a viral-associated, non-coding RNA (VA RNA), all operably linked to a second promoter; and (iii) a third vector comprising Rep and Cap, each operably linked to a third promoter; (b) culturing the transfected E1 complementary producer cell under conditions suitable for producing the AAV, wherein E1A is expressed at a ratio of about 1:1 to about 3:1 to each of E2A, E4, VA RNA, Rep, and Cap; and (c) purifying the AAV from the cultured E1 complementary producer cell, thereby obtaining the AAV. Suitably, one or more of the first, second, or third vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI.

In still further embodiments, the disclosure provides a method of producing an adeno-associated virus (AAV) in an E1 complementary producer cell, comprising: (a) transfecting the E1 complementary producer with: (i) a first vector comprising E1A, E2A, E4 and VA RNA, all operably linked to a first promoter; and (ii) a second vector comprising Rep and Cap, operably linked to a second promoter, wherein a copy number ratio of E1A to each of E2A, E4, VA RNA, Rep, and Cap is about 1:1 to about 3:1; (b) culturing the transfected E1 complementary producer cell under conditions suitable for producing the AAV; and (c) purifying the AAV from the cultured E1 complementary producer cell, thereby obtaining the AAV. Suitably, one or both of the first and second vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI.

### BRIEF DESCRIPTION OF THE DRAWINGS

The following drawings form part of the present specification and are included to further demonstrate exemplary embodiments of certain aspects of the present invention.

FIG. 1 shows the standard triple transfection plasmids for AAV production in E1 complementary producer cells, as described in embodiments herein: a pHelper plasmid containing E2A, E4, and VA RNA; a pRep-Cap plasmid containing Rep and Cap; and a pAAV-GOI plasmid containing inverted terminal repeat (ITR) sequences and a gene of interest (GOI).

FIG. 2 shows the viral titer results of a transfection experiment described in embodiments herein. HEK293 cells were transfected with the standard triple transfection plasmids (“3×Tnfx”) and one or more additional helper genes (empty vector (control), E1A alone, E1B-19k alone, E1B-55k alone, NP1-NS2, E1A+E1B-19k, E1A+19B-55k, E1B-19k+E1B-55k, and E1A+E1B-19k+E1B-55k). The crude virus titer was analyzed by droplet digital PCR (ddPCR).

FIG. 3A shows the viral titer results of a transfection experiment described in embodiments herein. HEK293 cells were transfected with the standard triple transfection plasmids (“3×Tnfx”) and one or more additional helper genes (empty vector (control), E1A alone, E1B-19k alone, E1B-55k alone, NP1-NS2, and E1A+NP1-NS2). The crude virus titer was analyzed by droplet digital PCR (ddPCR).

FIG. 3B shows the protein expression results from a transfection experiment described in embodiments herein. Western blot analysis was performed to evaluate the protein expression levels of E1A (289R, 243R, and 171R isoforms), Rep (Rep78 and Rep52 isoforms), and Cap (VP1, VP2, and VP3) in HEK293 cells indicated in FIG. 3A and in the Table. The levels of 3-actin were measured as a control.

FIG. 4A shows the protein expression results from a transfection experiment described in embodiments herein. The plasmid containing E1A was transfected at a 1:1, 2:1, or 3:1 molar ratio to each of the standard triple transfection plasmids (“3×Tnfx”), indicated as 1×E1A, 2×E1A, or 3×E1A, respectively. Western blot analysis was performed to evaluate the protein expression levels of E1A (289R, 243R, and 171R isoforms), Rep (Rep78 and Rep52 isoforms), and Cap (VP1, VP2, and VP3) in HEK293 cells transfected with the plasmids indicated in the Table.

FIG. 4B shows the viral titer results of a transfection experiment described in embodiments herein. HEK293 cells were transfected with plasmid containing E1A at a 1:1, 2:1, or 3:1 molar ratio to each of the standard triple transfection plasmids (“3×Tnfx”), indicated as 1×E1A, 2×E1A, or 3×E1A, respectively. The crude virus titer was analyzed by droplet digital PCR (ddPCR).

FIG. 5A shows the novel and enhanced triple transfection plasmids for AAV production in E1 complementary producer cells, as described in embodiments herein: a pLHI_Helper plasmid containing E1A, E2A, E4, and VA RNA; a pRep-Cap plasmid containing Rep and Cap; and a pAAV-GOI plasmid containing inverted terminal repeat (ITR) sequences and a gene of interest (GOI).

FIG. 5B shows the viral titer results of a transfection experiment as described in embodiments herein. HEK293 cells were transfected with the standard triple transfection plasmids (as shown in FIG. 1) containing pHelper, or the novel and enhanced triple transfection plasmids (as shown in FIG. 5) containing pLHI_Helper for AAV2 and AAV9 production. The crude virus titer was analyzed by droplet digital PCR (ddPCR).

### DETAILED DESCRIPTION OF THE INVENTION

Unless otherwise defined herein, scientific and technical terms used in the present disclosure shall have the meanings that are commonly understood by one of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The use of the term “or” in the claims is used to mean “and/or,” unless explicitly indicated to refer only to alternatives or the alternatives are mutually exclusive, although the disclosure supports a definition that refers to only alternatives and “and/or.”

As used herein, the terms “comprising” (and any variant or form of comprising, such as “comprise” and “comprises”), “having” (and any variant or form of having, such as “have” and “has”), “including” (and any variant or form of including, such as “includes” and “include”) or “containing” (and any variant or form of containing, such as “contains” and “contain”) are inclusive or open-ended and do not exclude additional, unrecited, elements or method steps.

The use of the term “for example” and its corresponding abbreviation “e.g.” (whether italicized or not) means that the specific terms recited are representative examples and embodiments of the disclosure that are not intended to be limited to the specific examples referenced or cited unless explicitly stated otherwise.

As used herein, “between” is a range inclusive of the ends of the range. For example, a number between x and y explicitly includes the numbers x and y, and any numbers that fall within x and y.

Throughout this application, the term “about” is used to indicate that a value includes the inherent variation of error for the method/device being employed to determine the value. Typically the term is meant to encompass approximately or less than 1%, 2%, 3%, 4%, 5%, 6%, 7%, 8%, 9%, 10%, 11%, 12%, 13%, 14%, 15%, 16%, 17%, 18%, 19% or 20% variability depending on the situation.

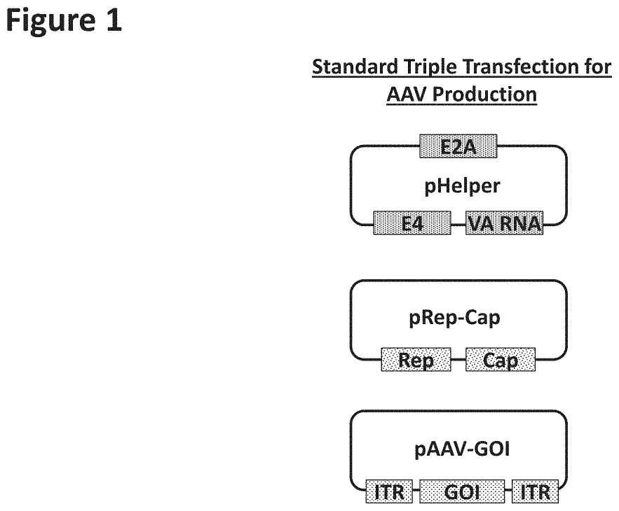

Adeno-associated virus (AAV) has emerged as the vector of choice for gene therapy in over 120 clinical trials worldwide. Improvement of the current standard for AAV production in E1 complementary producer cells, such as HEK293, PER.C6, or CAP® cells, is required to meet the fast-growing demand of recombinant AAV. As described herein, the standard AAV production method comprises the transfection of three plasmids into E1 complementary producer cells: pHelper containing E2A, E4, and VA; pRepCap containing Rep and Cap; and pAAV containing the gene of interest (GOI), termed the “standard triple transfection” and shown in FIG. 1. The E1 complementary producer cells natively express the remaining helper genes E1A and E1B, and thus, E1A and E1B are not included in pHelper.

It was therefore surprisingly discovered that transfection of E1A into E1 complementary producer cells, e.g., HEK293 cells, therefore providing additional E1A above that found natively, significantly improved AAV titer. The increase in titer was generally dose-dependent on the amount of E1A introduced into the E1 complementary producer cells. Thus, provided herein are methods for AAV production in E1 complementary producer cells with higher yields as compared to the current standard triple transfection method.

In embodiments, the disclosure provides a method of producing an adeno-associated virus (AAV) in an E1 complementary producer cell, comprising: (a) transfecting the E1 complementary producer cell with one or more vectors comprising: (i) an E1A adenovirus helper gene; (ii) an adenovirus helper gene selected from E2A, E4, or both; (iii) a viral-associated, non-coding RNA (VA RNA); and (iv) an AAV gene selected from Rep, Cap, or both; (b) culturing the transfected E1 complementary producer cell under conditions suitable for producing the AAV; and (c) purifying the AAV from the cultured E1 complementary producer cell, thereby obtaining the AAV.

As used herein, a “vector” or “expression vector” refers to a nucleic acid replicon, such as a plasmid, phage, virus, or cosmid, to which another nucleic acid segment may be attached to bring about the replication and/or expression of the attached nucleic acid segment in a host cell. The term “vector” includes episomal (e.g., plasmids) and non episomal vectors. The term “vector” may also include synthetic vectors. Non-limiting examples of vectors include baculovirus vectors, bacteriophage vectors, plasmids, phagemids, cosmids, fosmids, bacterial artificial chromosomes, viral vectors (e.g. viral vectors based on vaccinia virus, poliovirus, adenovirus, adeno-associated virus, SV40, herpes simplex virus, and the like), P1-based artificial chromosomes, yeast plasmids, yeast artificial chromosomes, and any other vectors specific for specific hosts of interest (e.g., mammalian cells such as the E1 complementary producer cells described herein, including but not limited to HEK293 cells, PER.C6 cells, CAP® cells, and derivatives thereof). Vectors may be introduced into the desired host cells, e.g., HEK293 cells, PER.C6 cells, CAP® cells, or derivatives thereof, by well-known methods, including, but not limited to, transfection, transduction, cell fusion, and lipofection. Vectors can comprise various regulatory elements including, e.g., constitutive and inducible promoters, transcription enhancers, transcription terminators, and the like.

As used herein, “transfection” means the introduction of an exogenous nucleic acid molecule, e.g., a vector, into a cell. A “transfected” cell comprises an exogenous nucleic acid molecule inside the cell, and a “transformed” cell is one in which the exogenous nucleic acid molecule within the cell induces a phenotypic change in the cell. The transfected nucleic acid molecule can be integrated into the host cell's genomic DNA and/or can be maintained by the cell, temporarily or for a prolonged period of time, extra-chromosomally. Host cells or organisms that express exogenous nucleic acid molecules or fragments are referred to as “recombinant,” “transformed,” or “transgenic” organisms. A number of transfection techniques are generally known in the art and include various chemical and physical methods, for example, electroporation, cell injection, calcium phosphate exposure, liposome or polymer-based carrier systems, and the like. See, e.g., Graham et al., Virology 52:456 (1973); Sambrook et al., Molecular Cloning, A Laboratory Manual, Cold Spring Harbor Laboratories, New York (1989); Davis et al., Basic Methods in Molecular Biology, Elsevier (1986); and Chu et al., Gene 13:197 (1981). Such techniques can be used to introduce one or more exogenous nucleic acid molecules, such as the one or more vectors for producing AAVs described herein, into suitable host cells, such as HEK293 cells.

A “nucleic acid,” “nucleic acid molecule,” “polynucleotide,” or “oligonucleotide” means a polymeric compound comprising covalently linked nucleotides. The term “nucleic acid” includes RNA and DNA, both of which may be single- or double-stranded. DNA includes, but is not limited to, complementary DNA (cDNA), genomic DNA, plasmid or vector DNA, and synthetic DNA. RNA includes, but is not limited to, mRNA, tRNA, rRNA, snRNA, microRNA, or miRNA.

In embodiments, the nucleic acids herein have at least 70%, at least 75%, at least 80%, at least 85%, at least 90%, at least 95%, at least 97%, at least 98%, at least 99%, or about 100% sequence identity with a reference nucleic acid (or a fragment of the reference nucleic acid). In embodiments, the polypeptides herein have at least 70%, at least 75%, at least 80%, at least 85%, at least 90%, at least 95%, at least 97%, at least 98%, at least 99%, or about 100% sequence identity with a reference polypeptide (or a fragment of the reference polypeptide). For example, when referring to the E1A gene, one of ordinary skill in the art would understand that the E1A gene encompasses a nucleic acid having at least 70%, at least 75%, %, at least 80%, at least 85%, at least 90%, at least 95%, at least 97%, at least 98%, at least 99%, or about 100% sequence identity with a wild-type adenovirus E1A gene, provided that the function of the E1A gene with respect to AAV production is not affected. As used herein, the terms “sequence identity” and “percent identity” in the context of nucleic acids, refer to the percentage of nucleotides that are the same when the nucleic acid sequences are aligned over a specified comparison window. Methods of alignment for determination of sequence identity are well-known can be performed using publicly available databases such as BLAST.

A “gene” refers to a nucleic acid that encode a polypeptide and includes cDNA and genomic DNA nucleic acid molecules. In some embodiments, “gene” also refers to a non-coding nucleic acid fragment that can act as a regulatory sequence preceding (i.e., 5′) and following (i.e., 3′) the coding sequence.

As used herein, the term “adeno-associated virus (AAV)” refers to a small sized, replicative-defective nonenveloped virus containing a single stranded DNA of the family Parvoviridae and the genus Dependoparvovirus. Over ten adeno-associated virus serotypes have been identified so far, with serotype AAV2 being the best characterized. Other non-limiting examples of AAV serotypes are ANC80, AAV1, AAV3, AAV4, AAV5, AAV6, AAV7, AAV8, AAV9, AAV10, and AAV11. In addition to these serotypes, AAV pseudotypes have been developed. An AAV pseudotype contains the capsid of a first serotype and the genome of a second serotype (e.g. the pseudotype AAV2/5 would correspond to an AAV with the genome of serotype AAV2 and the capsid of AAV5).

As used herein, the term “adenovirus” refers to a nonenveloped virus with an icosahedral nucleocapsid containing a double stranded DNA of the family Adenoviridae. Over 50 adenoviral subtypes have been isolated from humans and many additional subtypes have been isolated from other mammals and birds. These subtypes belong to the family Adenoviridae, which is currently divided into two genera, namely Mastadenovirus and Aviadenovirus. All adenoviruses are morphologically and structurally similar. In humans, however, adenoviruses show diverging immunological properties and are, therefore, divided into serotypes. Two human serotypes of adenovirus, namely Ad2 and Ad5, have been studied intensively and have provided the majority of general information about adenoviruses.

As used herein, the term “E1 complementary producer cell” refers to a cell line, typically a human cell line, that has the adenovirus E1A and E1B genes integrated or inserted into its genome. E1 complementary producer cells are capable of expressing E1A and E1B natively and are therefore suitable for AAV production without requiring exogenous E1A and/or E1B genes. The E1A and E1B genes may be suitably integrated in the E1 complementary producer cell as part of a longer sequence, e.g., containing additional adenovirus sequences, or the E1A and E1B genes may be suitably integrated in the E1 complementary producer cell without any additional adenovirus sequences, i.e., only the minimum required coding sequences for expressing E1A and E1B are integrated. Integration of adenovirus genes into mammalian cells, e.g., human cells, is known in the art. E1 complementary producer cells may be derived from any suitable cell line. Exemplary E1 complementary producer cells include, but are not limited to, HEK293 cells, 911 cells, pTG6559 cells, PER.C6 cells, GH329 cells, N52.E6 cells (also known as “CAP®” cells), HeLa-E1 cells, UR cells, VLI-293 cells, Ac51 cells, Ac139 cells, and variants and derivatives thereof. E1 complementary producer cells are further described in, e.g., Kovesdi et al., Viruses 2(8):1681-1703 (2010) and Farson et al., Mol Ther 14(2):305-311 (2006). In some embodiments, the E1 complementary producer cell of the present disclosure is a HEK293 cell, a PER.C6 cell, or a CAP® cell.

As used herein, the term “human embryonic kidney 293 (HEK293) cell” refers to a cell line originally derived from human embryonic kidney cells and containing approximately 4.5 kb of Ad5 genome, including the E1A and E1B genes. HEK293 cells are an exemplary E1 complementary producer cell as described herein. Variants of HEK293 cells have been developed and include, e.g., HEK293S, HEK293T, HEK293F, HEK293FT, HEK293FTM, HEK293SG, HEK293SGGD, HEK293H, HEK293E, HEK293MSR, and HEK293A, each of which has distinct characteristics, and all of which contain the E1A and E1B genes. The term “HEK293 cell” as used herein encompasses all HEK293 cell variants and derivatives, including but not limited to the variants described herein. For example, the HEK293T cell line expresses a temperature-sensitive allele of the SV40 T antigen, which enables the amplification of vectors containing the SV40 origin. See, e.g., Yuan et al., “The Scattered Twelve Tribes of HEK293,” Biomed Pharmacol J2018; 11(2).

As used herein, the term “PER.C6 cell” refers to a cell line derived from human embryonic retinal cells transformed with the E1 region of Ad5. PER.C6 cells natively express E1A and E1B and are an exemplary E1 complementary producer cell as described herein. The term “PER.C6 cell” as used herein encompasses all PER.C6 cell variants and derivatives. PER.C6 cells are further described in, e.g., Fallaux et al., Human Gene Ther 9(13):1909-1917 (1998).

As used herein, the term “CAP® cell” (also referred to herein as “CEVEC's Amniocyte Production cell” or “N52.E6 cell”) refers to a cell line derived from primary human amniocytes transformed with the E1 region of Ad5 (accessible under RRID:CVCL_IQ88). CAP® cells natively express E1A and E1B and are an exemplary E1 complementary producer cell as described herein. CAP® cell variants include, but are not limited to, CAP-T (accession ID: CVCL_WI61), which expresses the large T antigen of SV40. See, e.g., Wölfel et al., BMC Proceedings 5(Suppl 8):P133 (2011). The term “CAP® cell” as used herein encompasses all CAP® cell variants and derivatives.

As used herein, the term “E1A” refers to the adenovirus early region 1A (E1A) gene, which is expressed during adenovirus replication in the early phase of the viral life span. The E1A gene produces multiple protein isoforms, including but not limited to 289R, 243R, 217R, 171R, and 55R, with 289R, 243R, and 171R being the major isoforms of the Ad5 E1A gene. As used herein, the term “E1B” refers to the adenovirus early region 1B (E1B) gene, which expresses two proteins: a 55 kDa protein and a 19 kDa protein, termed “E1B-19k” and “E1B-55k,” respectively. As described herein, E1A, E1B-19k, and E1B-55k are adenovirus helper genes expressed by E1 complementary producer cells. As E1A, E1B-19k, and E1B-55k are produced by E1 complementary producer cells in sufficient amounts for AAV production, traditionally E1A and E1B genes are not exogenously introduced into E1 complementary producer cells when producing AAVs.

As used herein, the term “E2A” refers to the adenovirus E2A helper gene, which encodes a 72 kDa DNA-binding protein that regulates viral replication. As used herein, the term “E4” refers to the adenovirus E4 helper gene, which encodes proteins that modulate viral replication and protein expression, including E4 ORF3 and E4 ORF6. In embodiments, E4 comprises one or both of E4orf3 and E4orf6. As used herein, the term “viral-associated, non-coding RNA (VA RNA)” refers to an adenovirus non-coding RNA that plays a role in regulating translation and includes VAI (or VA_{I} or VA RNAI) and VAII (or VA₁₁ or VA RNAII). In embodiments, VA RNA comprises one or both of VAI and VAII. In the context of AAV production, the adenovirus genes E2A, E4, and VA RNA mediate AAV replication. As described herein, current methods for producing AAVs in E1 complementary producer cells include transfecting the E1 complementary producer cell with a pHelper plasmid that includes the E2A, E4, and VA RNA genes.

As used herein, the term “Rep” refers to an AAV coding region or functional homolog thereof that encodes the replication proteins of the virus, which are collectively required for replicating the viral genome. Functional homologs of the AAV Rep include, e.g., the Rep coding region from the human herpesvirus 6 (HHV-6), which is also known to mediate AAV DNA replication. The Rep coding region encodes at least the genes encoding for AAV Rep78, Rep68, Rep52, and Rep40, or functional homologs thereof. The Rep coding region may be derived from any AAV serotype, e.g., as described herein. The Rep coding region is not required to include all wild-type genes but may be altered (e.g., by insertion, deletion, or mutation of one or more nucleotides) so long as the Rep genes present provide for sufficient replication functions when expressed in the E1 complementary producer cell.

As used herein, the term “Cap” refers to an AAV coding region or functional homolog thereof that encodes the capsid proteins of the virus. Non-limiting examples of capsid proteins are the AAV capsid proteins VP1, VP2, and VP3. The Cap genes described herein can be derived from any AAV serotype or a combination of AAV serotypes.

In some embodiments, the E1 complementary producer cell is transfected with one or more vectors comprising one or more of (i) E1A, (ii) one or both of E2A and E4, (iii) VA RNA, and (iv) one or both of Rep and Cap. In certain embodiments, (i), (ii), (iii), and (iv) are on a single vector. In further embodiments, (i), (ii), (iii), and (iv) are on more than one vector. Suitably, (i), (ii), and (iii) can be on a first vector, and (iv) can be on a second vector. In embodiments, (ii) comprises both E2A and E4. In further embodiments, (iv) comprises both Rep and Cap. For example, a first vector can comprise E1A, E2A, E4, and VA RNA, and a second vector can comprise Rep and Cap. Suitably, (i) can be on a first vector, (ii) and (iii) can be on a second vector, and (iv) is on a third vector. For example, a first vector can comprise E1A, a second vector can comprise E2A, E4, and VA RNA, and a third vector can comprise Rep and Cap. Suitably, any one of (i), (ii), (iii), and (iv) can be on a separate vector, e.g., each of (i), (ii), (iii), and (iv) can be on a separate vector. For example, a first vector can comprise E1A, a second vector can comprise E2A and E4, a third vector can comprise VA RNA, and a fourth vector can comprise Rep and Cap. Suitably, the E1 complementary producer cell is a HEK293 cell, a PER.C6 cell, or a CAP® cell.

In some embodiments, E1A is transfected into the E1 complementary producer cell in a higher amount than any one of E2A, E4, VA RNA, Rep, and Cap. As discussed herein, it was unexpectedly discovered that AAV titers were dose-dependent on amount of E1A expressed in the E1 complementary producer cells. Thus, in embodiments, the E1A is introduced into the E1 complementary producer cell at a 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, or more than 10-fold higher amount than any of E2A, E4, VA RNA, Rep, and Cap. Methods of increasing expression of a particular gene can include, e.g., increasing the amount of the gene introduced into the cell and increasing the transcription level of the gene in the cell. Suitably, the E1 complementary producer cell is a HEK293 cell, a PER.C6 cell, or a CAP® cell.

Suitably, the vector comprising E1A can be transfected into the E1 complementary producer cell at a ratio of about 1:1 to about 10:1 to each of the vectors comprising E2A, E4, VA RNA, Rep, and/or Cap, or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1, or about 1:1 to about 2:1, or about 1:1 to about 1.5:1, to each of the vectors comprising E2A, E4, VA RNA, Rep, and/or Cap. In some embodiments, the ratio of the vector comprising E1A to each of the vectors comprising E2A, E4, VA RNA, Rep, and/or Cap transfected into the E1 complementary producer cell is 1:1. In further embodiments, the ratio of the vector comprising E1A to each of the vectors comprising E2A, E4, VA RNA, Rep, and/or Cap transfected into the E1 complementary producer cell is 2:1. In yet further embodiments, the ratio of the vector comprising E1A to each of the vectors comprising E2A, E4, VA RNA, Rep, and/or Cap transfected into the E1 complementary producer cell is 3:1. Suitably, the E1 complementary producer cell is a HEK293 cell, a PER.C6 cell, or a CAP® cell.

In some embodiments, the expression level of E1A in the E1 complementary producer cell (e.g., HEK293 cell, a PER.C6 cell, or a CAP® cell) is higher as compared to each of E2A, E4, VA RNA, Rep, and/or Cap. Suitably, each of E1A, E2A, E4, VA RNA, Rep, and/or Cap can be operably linked to one or more promoters. In some embodiments, each of E1A, E2A, E4, VA RNA, Rep, and/or Cap is operably linked to a separate promoter. As used herein, the terms “operably linked,” “in operable combination,” and “in operable order” mean that a polynucleotide of interest (e.g., E1A) is linked to a regulatory element (e.g., a promoter) in a manner that allows for expression of the polynucleotide of interest. As used herein, the terms “promoter,” “promoter sequence,” and “promoter region” refer to a polynucleotide capable of binding RNA polymerase and initiating transcription of a downstream coding or non-coding gene sequence. Promoters include constitutive promoters, which allow for continual transcription of its associated gene; and inducible promoters, which can be switched from an off state to an on state upon addition of an inducer molecule. Promoters can be classified as “strong” or “weak” based their affinity for RNA polymerase and/or sigma factor. A strong promoter has a high rate of transcription initiation as compared to a weak promoter. Non-limiting examples of promoters include spleen focus-forming virus (SFFV) promoter, the human polypeptide chain elongation factor (EF1α) promoter, the phosphoglycerate kinase (PGK) promoter, the ubiquitin C (UBC) promoter, the cytomegalovirus (CMV) promoter, the chicken beta-actin (CBA) promoter, the tetracycline-controlled transactivator system (also known as the “Tet-On” system or tTA-dependent system, or the reverse tetracycline-controlled transactivator system (also known as the “Tet-Off” system or rtTA-dependent system), the Rous sarcoma virus long terminal repeat (RSV) promoter, the mouse mammary tumor virus (MMTV) promoter, the actin promoter, the cytokeratin 14 promoter, or the cytokeratin 18 promoter. In some embodiments, the promoter operably linked to E1A is a stronger promoter as compared to the promoter(s) operably linked to any of E2A, E4, VA RNA, Rep, and Cap.

Suitably, the promoter operably linked to E1A can provide 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, or more than 10-fold higher expression as compared to the promoter(s) operably linked to any of E2A, E4, VA RNA, Rep, and Cap. In further embodiments, the promoter operably linked to E1A provides substantially a same expression level as compared to the promoter(s) operably linked to any of E2A, E4, VA RNA, Rep, and Cap. As used herein, “substantially similar” or “substantially the same” expression level means that the expression level is within 20%, within 15%, within 10%, within 5%, or within 1% of a reference expression level.

In some embodiments, E1A is operably linked to a promoter and further operably linked to an enhancer, and each of E2A, E4, VA RNA, Rep, and/or Cap is suitably not linked to an enhancer. As used herein, the term “enhancer” refers to a regulatory element that activate transcription to a higher level than would be the case in their absence, e.g., by recruiting transcription factors. Enhancers can activate promoter transcription over large distances (e.g., up to 1 Mbp away from the transcription start site) and independently of orientation. Non-limiting examples of enhancers include the CMV enhancer, α-fetoprotein MERII enhancer, MCK enhancer, or SV40 polyA enhancer. Suitably, the E1A, which is operably linked to a promoter and an enhancer, can provide higher expression levels as compared to E2A, E4, VA RNA, Rep, and/or Cap, which are not linked to enhancers.

Suitably, the one or more vectors can include an origin of replication to increase the number of plasmids produced by the host cell, which leads to increased levels of the proteins encoded by the genes on the one or more vectors. Exemplary origins of replication include, but are not limited to, the SV40 origin of replication, the Epstein-Barr (EBV) origin of replication, and others known to one of ordinary skill in the art.

In some embodiments, the number of copies of E1A, referred to herein as “copy number,” present on the one or more vectors is higher than the number of copies of each of E2A, E4, VA RNA, Rep, and/or Cap on the one or more vectors. In an example, a single vector can contain 2 or more copies of E1A and a single copy of each of E2A, E4, VA RNA, Rep, and Cap. In another example, for each copy of E2A, E4, VA RNA, Rep, and Cap present on the one or more vectors, at least 1, at least 2, at least 3, at least 4, at least 5, at least 6, at least 7, at least 8, at least 9, or at least 10 copies of E1A are present on the one or more vectors. Suitably, the copy number ratio of E1A to each of E2A, E4, VA RNA, Rep, and/or Cap on the one or more vectors can be about 1:1 to about 10:1, or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1, or about 1:1 to about 2:1, or about 1:1 to about 1.5:1. In some embodiments, the copy number ratio of E1A to each of E2A, E4, VA RNA, Rep, and/or Cap on the one or more vectors is about 1:1. In further embodiments, the copy number ratio of E1A to each of E2A, E4, VA RNA, Rep, and/or Cap on the one or more vectors is about 2:1. In yet further embodiments, the copy number ratio of E1A to each of E2A, E4, VA RNA, Rep, and/or Cap on the one or more vectors is about 3:1.

In some embodiments, the method comprises transfecting an additional helper gene into the E1 complementary producer cell. Thus, in embodiments, the one or more vectors further comprises (i) E1A, (ii) one or both of E2A and E4, (iii) VA RNA, (iv) one or both of Rep and Cap; and (v) a helper gene selected from E1B, NP1, NS2, or a combination thereof. Suitably, the E1 complementary producer cell is a HEK293 cell, a PER.C6 cell, or a CAP® cell.

As discussed herein, E1B is an adenovirus helper gene and encodes the E1B-19k and E1B-55k helper proteins for AAV production. As used herein the terms “NP1” and “NS2” refer to human bocavirus (HBoV1) genes that encode the NP1 and NS2 nonstructural proteins, respectively. NP1 and NS2 have been discovered as helper genes for AAV replication and were shown to enhance AAV titers when co-expressed with the conventional adenovirus helper genes for E1 complementary producer cells, i.e., E2A, E4, and VA RNA as described herein. See, e.g., Wang et al., Mol Ther Methods Clin Dev 11:40-51 (2018). In some embodiments, transfecting one or more of E1B, NP1, and NS2, in combination with (i), (ii), (iii), and (iv) as described herein, further increases AAV titers in E1 complementary producer cells. In certain embodiments, (v) comprises EB1. In further embodiments, (v) comprises NP1 and NS2. In yet further embodiments, (v) comprises E1B, NP1, and NS2. Suitably, the E1 complementary producer cell is a HEK293 cell, a PER.C6 cell, or a CAP® cell.

In some embodiments, (i), (ii), (iii), (iv), and (v) are on a single vector. In further embodiments, (i), (ii), (iii), (iv), and (v) are on more than one vector. For example, (i), (ii), and (iii) can be on a first vector, (iv) can be on a second vector, and (v) can be on a third vector. In another example, (i), (ii), (iii), and (v) can be on a first vector, and (iv) can be on a second vector.

In a further example, (i), (ii), and (iii) can be on a first vector, and (iv) and (v) can be on one or more additional vectors. In a yet further example, (i) and (v) can be on a first vector, and (ii), (iii), and (iv) can be on one or more additional vectors. Suitably, any one of (i), (ii), (iii), (iv), and (v) can be on a separate vector, e.g., each of (i), (ii), (iii), (iv), and (v) can be on a separate vector. In embodiments, a first vector comprises E1A, E2A, E4, and VA RNA, and a second vector comprises Rep and Cap. In additional embodiments, a first vector comprises E1A, E2A, E4, and VA RNA, a second vector comprises Rep and Cap, and a third vector comprises E1B, NP1, NS2, or a combination thereof.

The E1 complementary producer cells (e.g., HEK293 cells, PER.C6 cells, or CAP® cells) can be cultured, following transfection of (i) E1A, (ii) one or both of E2A and E4, (iii) VA RNA, (iv) one or both of Rep and Cap; (v) a helper gene selected from E1B, NP1, NS2, or a combination thereof, and/or the GOI, using any suitable device, facility, and method described herein. Suitably, expression of (i), (ii), (iii), (iv), and (v) can be induced during the culturing. In certain embodiments, the promoter for any of (i), (ii), (iii), (iv), and/or (v) is a constitutive promoter. In further embodiments, the promoter for any of (i), (ii), (iii), (iv), and/or (v) is an inducible promoter, requiring an inducer molecule to activate transcription as described herein. In one aspect, the expression level of a gene under the control of an inducible promoter can be modulated by regulating the amount of the inducer molecule. In another aspect, the same inducible promoter can be regulated using more than one inducer molecule to effect different expression levels. In a further aspect, the promoter operably linked to (i) is a stronger promoter than the promoter(s) operably linked to (ii), (iii), (iv), and/or (v). For example, (i) can be under control of a relatively strong promoter such as the CMV or SV40 promoter, and (ii), (iii), (iv), and/or (v) can be under control of a relatively weaker promoter such as the UBC or PGK promoter. In a further aspect, the promoter operably linked to (i) is induced by a different inducer molecule than the promoter operably linked to (ii), (iii), (iv), and/or (v). For example, (i) can be under control of a Tet-On system that is inducible by tetracycline (Tet) or an analog such as doxycycline (Dox); (ii), (iii), (iv), and/or (v) can be under control of a constitutive promoter such as CMV, SV40, UBC, or PGK; and the level of E1A expression can be regulated by the amount of inducer molecule (e.g., Tet or Dox) added to the cell culture. Thus, the amount of inducer molecule added to the cell culture can determines the relative amounts of (i), (ii), (iii), (iv), and/or (v) expressed in the cell culture.

In embodiments where the one or more vectors comprise (i), (ii), (iii), and (iv), the culturing comprises regulating the expression of (i), (ii), (iii), and (iv), such that E1A is expressed at a ratio of 1:1 to about 10:1 to each of (ii), (iii), and (iv), or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1, or about 1:1 to about 2:1, or about 1:1 to about 1.5:1 to each of (ii), (iii), and (iv). Suitably, the ratio of the expression of E1A to the expression of each of (ii), (iii), and (iv) is about 1:1. In further embodiments, the ratio of the expression of E1A to the expression of each of (ii), (iii), and (iv) is about 2:1. In yet further embodiments, the ratio of the expression of E1A to the expression of each of (ii), (iii), and (iv) is about 3:1.

In embodiments where the one or more vectors comprises (i), (ii), (iii), (iv), and (v), the culturing comprises regulating the expression of (i), (ii), (iii), (iv), and (v), such that E1A is expressed at a ratio of 1:1 to about 10:1, or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1, or about 1:1 to about 2:1, or about 1:1 to about 1.5:1 to each of (ii), (iii), (iv), and (v). Suitably, the ratio of the expression of E1A to the expression of each of (ii), (iii), (iv), and (v) is about 1:1, or about 2:1, or about 3:1.

In some embodiments, the one or more vectors further comprises a gene of interest (GOI). Suitably, the GOI is on a separate vector from each of (i), (ii), (iii), (iv), and (v). Thus, in embodiments, the method further comprises transfecting the E1 complementary producer cell with a vector comprising a GOI, wherein the GOI is on a separate vector from each of (i), (ii), (iii), (iv), and (v). Suitably, the vector comprising (i) is transfected into the E1 complementary producer cell at a ratio of about 1:1 to about 10:1 to the vector comprising the GOI, or about 1:1 to about 9:1, or about 1:1 to about 8:1, or about 1:1 to about 7:1, or about 1:1 to about 6:1, or about 1:1 to about 5:1, or about 1:1 to about 4:1, or about 1:1 to about 3:1, or about 1:1 to about 2:1, or about 1:1 to about 1.5:1 to the vector comprising the GOI. Suitably, the ratio of the vector comprising (i) to the ratio of the vector comprising the GOI transfected into the E1 complementary producer cell is about 1:1, about 2:1, or about 3:1. Suitably, the E1 complementary producer cell is a HEK293 cell, a PER.C6 cell, or a CAP® cell.

The vector comprising the GOI can include two inverted terminal repeat (ITR) sequences that flank the GOI. As known in the art, these ITR sequences are single-stranded nucleotide sequences followed downstream by its reverse complement. ITR sequences represent the minimal sequence required for replication, rescue, packaging, and integration of the AAV genome.

In general, as used herein, the term “GOI” refers to a heterologous gene, i.e., a gene that is not normally joined together and/or not normally associated with a particular host cell. In some embodiments, a heterologous gene is a construct where the coding sequence itself is not found in nature (e.g., a synthetic sequence having codons different from the native gene). Suitably, the GOI can be a reporter gene, a selection gene, or a gene of therapeutic interest.

As referred to herein, a “reporter gene” is a gene that, upon expression, confers a phenotype upon a cell that can be easily identified and measured. In some embodiments, the reporter gene encodes a fluorescent protein. In further embodiments, the reporter gene comprises a selection gene.

As referred to herein, a “selection gene” is a gene that encodes a protein having an enzymatic activity, which confers to the host cell the ability to grow in medium lacking an otherwise essential nutrient. Alternatively or additionally, a selection gene may confer resistance to an antibiotic or drug upon the host cell in which the selection gene is expressed. A selection gene may be used to confer a particular phenotype upon the host cell. When a host cell expresses a selection gene to grow in selective medium, the gene is said to be a positive selection gene. A selection gene can also be used to select against host cells containing a particular gene; a selection gene used in this manner is referred to as a negative selection gene.

As used herein, the term “gene of therapeutic interest” refers to any functionally relevant nucleotide sequence. Thus, the gene of therapeutic interest of the present disclosure can comprise any desired gene that encodes a protein that is defective or missing from a target cell genome or that encodes a non-native protein having a desired biological or therapeutic effect (e.g., an antiviral function), or the sequence can correspond to a molecule having an antisense or ribozyme function. Representative (non-limiting) examples of suitable genes of therapeutic interest include those used for the treatment of inflammatory diseases, autoimmune, chronic and infectious diseases, including such disorders as AIDS, cancer, neurological diseases, cardiovascular disease, hypercholesterolemia; various blood disorders including various anemias, thalassemia and hemophilia; genetic defects such as cystic fibrosis, Gaucher's Disease, adenosine deaminase (ADA) deficiency, emphysema, etc. Several antisense oligonucleotides (e.g., short oligonucleotides complementary to sequences around the translational initiation site (AUG codon) of an mRNA) that are useful in antisense therapy for cancer and for viral diseases have been described in the art and are also examples of suitable genes of therapeutic interest.

In additional embodiments, the disclosure provides a method of producing an adeno-associated virus (AAV) in an E1 complementary producer cell, comprising: (a) transfecting the E1 complementary producer cell with: (i) a first vector comprising E1A operably linked to a first promoter; (ii) a second vector comprising E2A, E4, and a viral-associated, non-coding RNA (VA RNA), all operably linked to a second promoter; and (iii) a third vector comprising Rep and Cap, both operably linked to a third promoter, wherein a transfection ratio of (i) to each of (ii) and (iii) is about 1:1 to about 3:1; (b) culturing the transfected E1 complementary producer cell under conditions suitable for producing the AAV; and (c) purifying the AAV from the cultured E1 complementary producer cell, thereby obtaining the AAV. Suitably, one or more of the first, second, or third vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI. Suitably, the E1 complementary producer cell is a HEK293 cell, a PER.C6 cell, or a CAP® cell.

In further embodiments, the disclosure provides a method of producing an adeno-associated virus (AAV) in an E1 complementary producer cell, comprising: (a) transfecting the E1 complementary producer cell with: (i) a first vector comprising E1A operably linked to a first promoter and an enhancer; (ii) a second vector comprising E2A, E4, and a viral-associated, non-coding RNA (VA RNA), all operably linked to a second promoter; and (iii) a third vector comprising Rep and Cap, each operably linked to a third promoter; (b) culturing the transfected E1 complementary producer cell under conditions suitable for producing the AAV, wherein E1A is expressed at a ratio of about 1:1 to about 3:1 to each of E2A, E4, VA RNA, Rep, and Cap; and (c) purifying the AAV from the cultured E1 complementary producer cell, thereby obtaining the AAV. Suitably, one or more of the first, second, or third vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI. Suitably, the E1 complementary producer cell is a HEK293 cell, a PER.C6 cell, or a CAP® cell.

In still further embodiments, the disclosure provides a method of producing an adeno-associated virus (AAV) in an E1 complementary producer cell, comprising: (a) transfecting the E1 complementary producer cell with: (i) a first vector comprising E1A, E2A, E4 and VA RNA, all operably linked to a first promoter; and (ii) a second vector comprising Rep and Cap, operably linked to a second promoter, wherein a copy number ratio of E1A to each of E2A, E4, VA RNA, Rep, and Cap is about 1:1 to about 3:1; (b) culturing the transfected E1 complementary producer cell under conditions suitable for producing the AAV; and (c) purifying the AAV from the cultured E1 complementary producer cell, thereby obtaining the AAV. Suitably, one or both of the first and second vectors further comprises a helper gene selected from E1B, NP1, NS2, or a combination thereof. Suitably, the E1B is E1B-19k or E1B-55k. Suitably, the helper gene comprises a combination of NP1 and NS2. In embodiments, the method further comprises transfecting the E1 complementary producer cell with a vector comprising a GOI. Suitably, the E1 complementary producer cell is a HEK293 cell, a PER.C6 cell, or a CAP® cell.

In embodiments, provided herein is a method of treatment in a subject in need thereof with an AAV comprising: transfecting a E1 complementary producer cell (e.g., HEK293 cell, PER.C6 cell, or CAP® cell) with one or more vectors comprising: (i) an E1A adenovirus helper gene; (ii) an adenovirus helper gene selected from E2A, E4, or both; (iii) a viral-associated, non-coding RNA (VA RNA); and (iv) an AAV gene selected from Rep, Cap, or both; producing the AAV; harvesting the AAV; and administering the AAV to the subject. Methods of producing the AAV are further described herein. Suitably, the methods are used to treat the subject with a GOI, e.g., a gene of therapeutic interest. Administration to a human subject can include, for example, inhalation, injection, or intravenous administration, as well as other administration methods known in the art.

As discussed herein, methods of the present disclosure advantageously provide a higher titer of the AAV produced by the E1 complementary producer cells, as compared to a method that does not comprise transfecting the E1 complementary producer cell with E1A. In embodiments, the methods of the present disclosure provide at least 1.1-fold, at least 1.2-fold, at least 1.3-fold, at least 1.4-fold, at least 1.5-fold, at least 1.6-fold, at least 1.7-fold, at least 1.8-fold, at least 1.9-fold, at least 2-fold, at least 3-fold, at least 4-fold, at least 5-fold, at least 6-fold, at least 7-fold, at least 8-fold, at least 9-fold, or at least 10-fold or higher titer of the AAV as compared to a method that does not comprise transfecting the E1 complementary producer cell with E1A. Suitably, the E1 complementary producer cell is a HEK293 cell, a PER.C6 cell, or a CAP® cell.

The methods of producing the AAVs can be used in a continuous manufacturing system. Culturing conditions for E1 complementary producer cells, e.g., HEK293 cells, PER.C6 cells, or CAP® cells, are known in the field. In general, HEK293 cells, PER.C6 cells, and CAP® cells are suspension cultures, which are grown in culture flasks or large suspension vats that allow for a large surface area for gas and nutrient exchange. Suspension cell cultures often utilize a stirring or agitation mechanism to provide appropriate mixing. Media and conditions for maintaining cells in suspension are known to one of ordinary skill in the art. In exemplary embodiments, the use of a suspension cell culture allows for the production of large volumes of AAV, with high productivity and prolonged culture conditions to allow for multiple harvests of AAVs for each batch of starting cells.

Production methods can utilize any suitable reactor(s) including but not limited to stirred tank, airlift, fiber, microfiber, hollow fiber, ceramic matrix, fluidized bed, fixed bed, and/or spouted bed bioreactors. As used herein, “reactor” can include a fermenter or fermentation unit, or any other reaction vessel and the term “reactor” is used interchangeably with “fermenter.” For example, in some aspects, an example bioreactor unit can perform one or more, or all, of the following: feeding of nutrients and/or carbon sources, injection of suitable gas (e.g., oxygen), inlet and outlet flow of fermentation or cell culture medium, separation of gas and liquid phases, maintenance of temperature, maintenance of oxygen and CO2 levels, maintenance of pH level, agitation (e.g., stirring), and/or cleaning/sterilizing. Example reactor units, such as a fermentation unit, may contain multiple reactors within the unit, for example the unit can have 1, 2, 3, 4, 5, 10, 15, 20, 25, 30, 35, 40, 45, 50, 60, 70, 80, 90, or 100, or more bioreactors in each unit and/or a facility may contain multiple units having a single or multiple reactors within the facility. In various embodiments, the bioreactor can be suitable for batch, semi fed-batch, fed-batch, perfusion, and/or a continuous fermentation processes. Any suitable reactor diameter can be used. In embodiments, the bioreactor can have a volume between about 100 mL and about 50,000 L. Non-limiting examples include a volume of 100 mL, 250 mL, 500 mL, 750 mL, 1 liter, 2 liters, 3 liters, 4 liters, 5 liters, 6 liters, 7 liters, 8 liters, 9 liters, 10 liters, 15 liters, 20 liters, 25 liters, 30 liters, 40 liters, 50 liters, 60 liters, 70 liters, 80 liters, 90 liters, 100 liters, 150 liters, 200 liters, 250 liters, 300 liters, 350 liters, 400 liters, 450 liters, 500 liters, 550 liters, 600 liters, 650 liters, 700 liters, 750 liters, 800 liters, 850 liters, 900 liters, 950 liters, 1000 liters, 1500 liters, 2000 liters, 2500 liters, 3000 liters, 3500 liters, 4000 liters, 4500 liters, 5000 liters, 6000 liters, 7000 liters, 8000 liters, 9000 liters, 10,000 liters, 15,000 liters, 20,000 liters, and/or 50,000 liters. Additionally, suitable reactors can be multi-use, single-use, disposable, or non-disposable and can be formed of any suitable material including metal alloys such as stainless steel (e.g., 316 L or any other suitable stainless steel) and Inconel, plastics, and/or glass.

In embodiments and unless stated otherwise herein, the devices, facilities, and methods described herein can also include any suitable unit operation and/or equipment not otherwise mentioned, such as operations and/or equipment for separation, purification, and isolation of such products. Any suitable facility and environment can be used, such as traditional stick-built facilities, modular, mobile and temporary facilities, or any other suitable construction, facility, and/or layout. For example, in some embodiments modular clean-rooms can be used. Additionally and unless otherwise stated, the devices, systems, and methods described herein can be housed and/or performed in a single location or facility or alternatively be housed and/or performed at separate or multiple locations and/or facilities.

All references cited herein, including patents, patent applications, papers, textbooks and the like, and the references cited therein, to the extent that they are not already, are hereby incorporated herein by reference in their entirety.

### ADDITIONAL EXEMPLARY EMBODIMENTS

Embodiment 1 is a method of producing an adeno-associated virus (AAV) in an E1 complementary producer cell, comprising: (a) transfecting the E1 complementary producer cell with one or more vectors comprising: (i) an E1A adenovirus helper gene; (ii) an adenovirus helper gene selected from E2A, E4, or both; (iii) a viral-associated, non-coding RNA (VA RNA); and (iv) an AAV gene selected from Rep, Cap, or both; (b) culturing the transfected E1 complementary producer cell under conditions suitable for producing the AAV; and (c) purifying the AAV from the cultured E1 complementary producer cell, thereby obtaining the AAV.

Embodiment 2 includes the method of embodiment 1, wherein (i), (ii), (iii), and (iv) are on a single vector.

Embodiment 3 includes the method of embodiment 1, wherein (i), (ii), (iii), and (iv) are on more than one vector.

Embodiment 4 includes the method of embodiment 3, wherein (i), (ii), and (iii) are on a first vector, and (iv) is a second vector.

Embodiment 5 includes the method of embodiment 3, wherein (i) is on a first vector, (ii) and (iii) are on a second vector, and (iv) is on a third vector.

Embodiment 6 includes the method of embodiment 3, wherein each of (i), (ii), (iii), and (iv) is on a separate vector.

Embodiment 7 includes the method of embodiment 5 or 6, wherein the vector comprising (i) is transfected into the E1 complementary producer cell at a ratio of about 1:1 to about 5:1 to each of the vectors comprising (ii), (iii), and (iv).

Embodiment 8 includes the method of embodiment 7, wherein the ratio of the vector comprising (i) to each of the vectors comprising (ii), (iii), and (iv) transfected into the E1 complementary producer cell is 1:1.

Embodiment 9 includes the method of embodiment 7, wherein the ratio of the vector comprising (i) to each of the vectors comprising (ii), (iii), and (iv) transfected into the E1 complementary producer cell is 2:1.

Embodiment 10 includes the method of embodiment 7, wherein the ratio of the vector comprising (i) to each of the vectors comprising of (ii), (iii), and (iv) transfected into the E1 complementary producer cell is 3:1.

Embodiment 11 includes the method of any one of embodiments 1 to 10, wherein (i), (ii), (iii), and (iv) are operably linked to one or more promoters.

Embodiment 12 includes the method of embodiment 11, wherein each of (i), (ii), (iii), and (iv) is operably linked to a separate promoter.

Embodiment 13 includes the method of embodiment 12, wherein the promoter operably linked to (i) provides substantially a same expression level as compared to the promoters operably linked to (ii), (iii), and (iv).

Embodiment 14 includes the method of embodiment 12, wherein the promoter operably linked to (i) provides at least 2-fold higher expression as compared to the promoters operably linked to (ii), (iii), and (iv).

Embodiment 15 includes the method of embodiment 12, wherein the promoter operably linked to (i) provides at least 3-fold higher expression as compared to the promoters operably linked to (ii), (iii), and (iv).

Embodiment 16 includes the method of any one of embodiments 11 to 15, wherein (i) is operably linked to a promoter and further operably linked to an enhancer, and wherein (ii), (iii), and (iv) are not operably linked to an enhancer.

Embodiment 17 includes the method of any one of embodiments 1 to 16, wherein a copy number ratio of (i) to each of (ii), (iii), and (iv) on the one or more vectors is about 1:1 to about 5:1.

Embodiment 18 includes the method of embodiment 17, wherein the copy number ratio of (i) to each of (ii), (iii), and (iv) on the one or more vectors is 1:1.

Embodiment 19 includes the method of embodiment 17, wherein the copy number ratio of (i) to each of (ii), (iii), and (iv) on the one or more vectors is 2:1.

Embodiment 20 includes the method of embodiment 17, wherein the copy number ratio of (i) to each of (ii), (iii), and (iv) on the one or more vectors is 3:1.

Embodiment 21 includes the method of any one of embodiments 1 to 20, wherein (ii) comprises E2A and E4.

Embodiment 22 includes the method of any one of embodiments 1 to 21, wherein (iv) comprises Rep and Cap.

Embodiment 23 includes the method of any one of embodiments 1 to 22, wherein the culturing comprises expressing E1A at a ratio of about 1:1 to about 5:1 to each of (ii), (iii), and (iv).

Embodiment 24 includes the method of embodiment 23, wherein the ratio of the expression of E1A to the expression of each of (ii), (iii), and (iv) is 1:1, 2:1, or 3:1.

Embodiment 25 includes the method of any one of embodiments 1 to 24, wherein the one or more vectors further comprises (v) a helper gene selected from E1B, NP1, NS2, or a combination thereof.

Embodiment 26 includes the method of embodiment 25, wherein the E1B is E1B-19k or E1B-55k.

Embodiment 27 includes the method of embodiment 25, wherein (v) comprises NP1 and NS2.

Embodiment 28 includes the method of any one of embodiments 25 to 27, wherein (i) and (v) are on a single vector.

Embodiment 29 includes the method of any one of embodiments 25 to 27, wherein (v) is on a separate vector from (i), (ii), (iii), and (iv).

Embodiment 30 includes the method of any one of embodiments 1 to 29, wherein the one or more vectors further comprises a gene of interest.

Embodiment 31 includes the method of any one of embodiments 1 to 29, further comprising transfecting the E1 complementary producer cell with a vector comprising a gene of interest, wherein the gene or interest is on a separate vector from (i), (ii), (iii), and (iv).

Embodiment 32 includes the method of any one of embodiments 1 to 31, wherein the method produces at least 1.5-fold or higher titer of the AAV as compared to a method that does not comprise transfecting an E1 complementary producer cell with an E1A adenovirus helper gene.

Embodiment 33 is a method of producing an adeno-associated virus (AAV) in an E1 complementary producer cell, comprising: (a) transfecting the E1 complementary producer cell with: (i) a first vector comprising E1A operably linked to a first promoter; (ii) a second vector comprising E2A, E4, and a viral-associated, non-coding RNA (VA RNA), all operably linked to a second promoter; and (iii) a third vector comprising Rep and Cap, both operably linked to a third promoter, wherein a transfection ratio of (i) to each of (ii) and (iii) is about 1:1 to about 3:1; (b) culturing the transfected E1 complementary producer cell under conditions suitable for producing the AAV; and (c) purifying the AAV from the cultured E1 complementary producer cell, thereby obtaining the AAV.

Embodiment 34 is a method of producing an adeno-associated virus (AAV) in an E1 complementary producer cell, comprising: (a) transfecting the E1 complementary producer cell with: (i) a first vector comprising E1A operably linked to a first promoter and an enhancer; (ii) a second vector comprising E2A, E4, and a viral-associated, non-coding RNA (VA RNA), all operably linked to a second promoter; and (iii) a third vector comprising Rep and Cap, each operably linked to a third promoter; culturing the transfected E1 complementary producer cell under conditions suitable for producing the AAV, wherein E1A is expressed at a ratio of about 1:1 to about 3:1 to each of E2A, E4, VA RNA, Rep, and Cap; and purifying the AAV from the cultured E1 complementary producer cell, thereby obtaining the AAV.

Embodiment 35 is a method of producing an adeno-associated virus (AAV) in an E1 complementary producer cell, comprising: (a) transfecting the E1 complementary producer cell with: (i) a first vector comprising E1A, E2A, E4 and VA RNA, all operably linked to a first promoter; and (ii) a second vector comprising Rep and Cap, operably linked to a second promoter, wherein a copy number ratio of E1A to each of E2A, E4, VA RNA, Rep, and Cap is about 1:1 to about 3:1; (b) culturing the transfected E1 complementary producer cell under conditions suitable for producing the AAV; and (c) purifying the AAV from the cultured E1 complementary producer cell, thereby obtaining the AAV.

Embodiment 36 includes the method of embodiment 33 or 34, wherein one or more of the first, second, or third vectors further comprises a helper gene selected from E1B, NP1, NS2, or a combination thereof.

Embodiment 37 includes the method of embodiment 35, wherein one or both of the first or second vectors further comprises a helper gene selected from E1B, NP1, NS2, or a combination thereof.

Embodiment 38 includes the method of any one of embodiments 33 to 35, further comprising transfecting the E1 complementary producer cell with a vector comprising helper gene selected from E1B, NP1, NS2, or a combination thereof.

Embodiment 39 includes the method of any one of embodiments 36 to 38, wherein the E1B is E1B-19k or E1B-55k.

Embodiment 40 includes the method of any one of embodiments 36 to 38, wherein the helper gene comprises a combination of NP1 and NS2.

Embodiment 41 includes the method of any one of embodiments 33 to 40, further comprising transfecting the E1 complementary producer cell with a vector comprising a gene of interest.

Embodiment 42 includes the method of any one of embodiments 1 to 41, wherein the E1 complementary producer cell is a HEK293 cell, a 911 cell, a pTG6559 cell, a PER.C6 cell, a GH329 cell, an N52.E6 (CAP®) cell, a HeLa-E1 cell, an UR cell, a VLI-293 cell, an Ac51 cell, an Ac139 cell, or a variant or derivative. thereof.

Embodiment 43 includes the method of embodiment 42, wherein the E1 complementary producer cell is a HEK293 cell, a PER.C6 cell, or a N52.E6 (CAP®) cell.

Embodiment 44 includes the method of embodiment 43, wherein the E1 complementary producer cell is a HEK293 cell.

Embodiment 45 includes the method of embodiment 44, wherein the HEK293 cell is a HEK293S cell, a HEK293T cell, a HEK293F cell, a HEK293FT cell, a HEK293FTM cell, a HEK293SG cell, a HEK293SGGD cell, a HEK293H cell, a HEK293E cell, a HEK293MSR cell, a HEK293A cell, or a combination thereof.

Embodiment 46 includes the method of embodiment 45, wherein the HEK293 cell is a HEK293T cell.

Embodiment 47 includes the method of embodiment 43, wherein the E1 complementary producer cell is a PER.C6 cell.

Embodiment 48 includes the method of embodiment 43, wherein the E1 complementary producer cell is a N52.E6 (CAP®) cell.

### EXAMPLES

### Example 1. Screening of Candidate Helper Genes for AAV Production

To identify new helper genes for improving AAV production by triple transfection, the full length cDNA for E1A, E1B-19k, E1B-55k, and human bocavirus NP1 and NS2 were synthesized. The cDNA for the additional helper genes were subcloned between the CMV promoter and the bovine growth hormone polyadenylation (BGH poly(A)) signal in the pcDNA3.1 vector to allow overexpression. NP1 and NS2 were linked with an internal ribosome entry site (IRES) for dual expression in a single mRNA transcript. The candidate plasmids contained: E1A alone (SEQ ID NO:1), E1B-19k alone (SEQ ID NO:2), E1B-55k alone (SEQ ID NO:3), NP1-NS2 (SEQ ID NO:4), E1A+E1B-19k, E1A+19B-55k, E1B-19k+E1B-55k, or E1A+E1B-19k+E1B-55k.

To test these candidate plasmids, HEK293 cells were grown in 30 mL culture in 125 mL shaker flasks and transfected with the standard triple transfection plasmids shown in FIG. 1 and a candidate helper plasmid. For the Rep/Cap plasmid, the pRep2-Cap9 was used, and for the pAAV plasmid, the pAAV-CMV-GFP was used for the production of AAV9.GFP viruses. The same amount of empty vector pcDNA3.1 was co-transfected as a control. Each candidate helper plasmid was tested in duplicate.

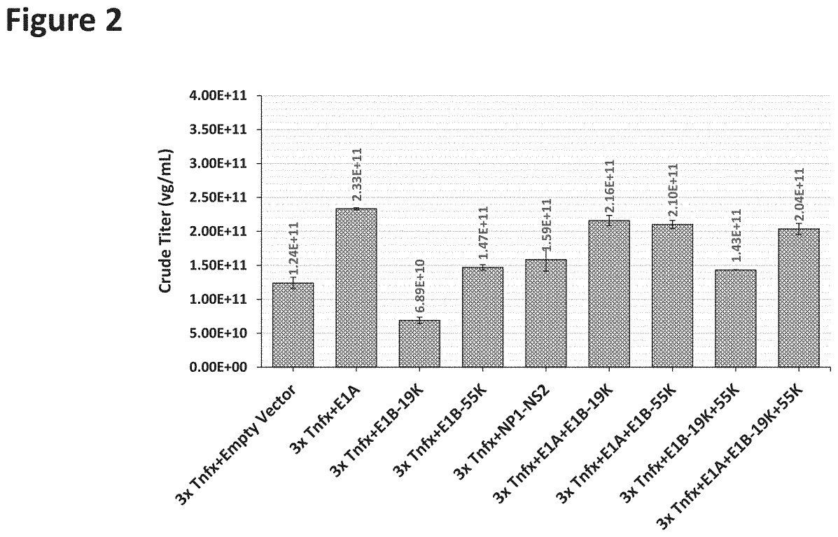

The crude viruses were harvested three days after transfection and subjected to droplet digital PCR (ddPCR) titer analysis to quantify the viral genome containing particles per milliliter (vg/mL). The results in FIG. 2 showed that the additional expression of E1A, or E1A plus E1B isoforms, significantly increased the titer by nearly 100% as compared to the control, while expression of the E1B isoforms or the bocavirus NP1 and NS2 had minimal improvement for the AAV titer.

### Example 2. Confirmation of E1A Over-Expression for AAV Production

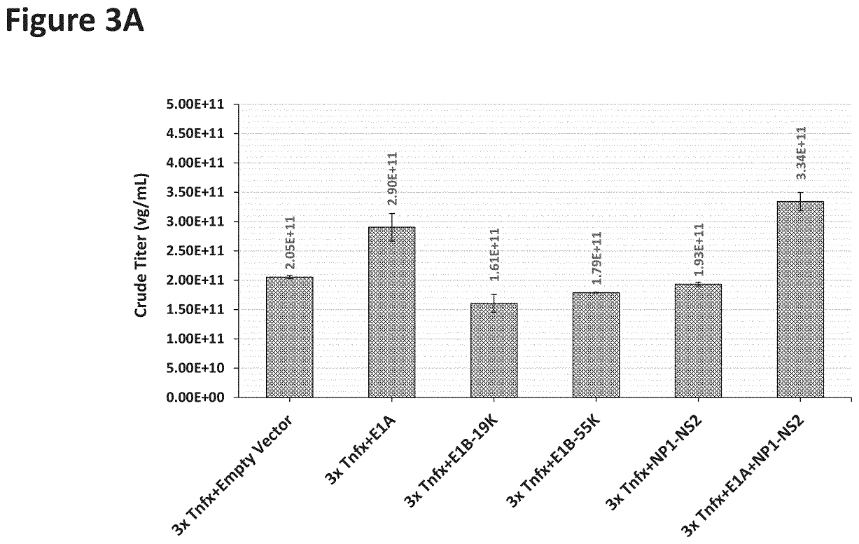

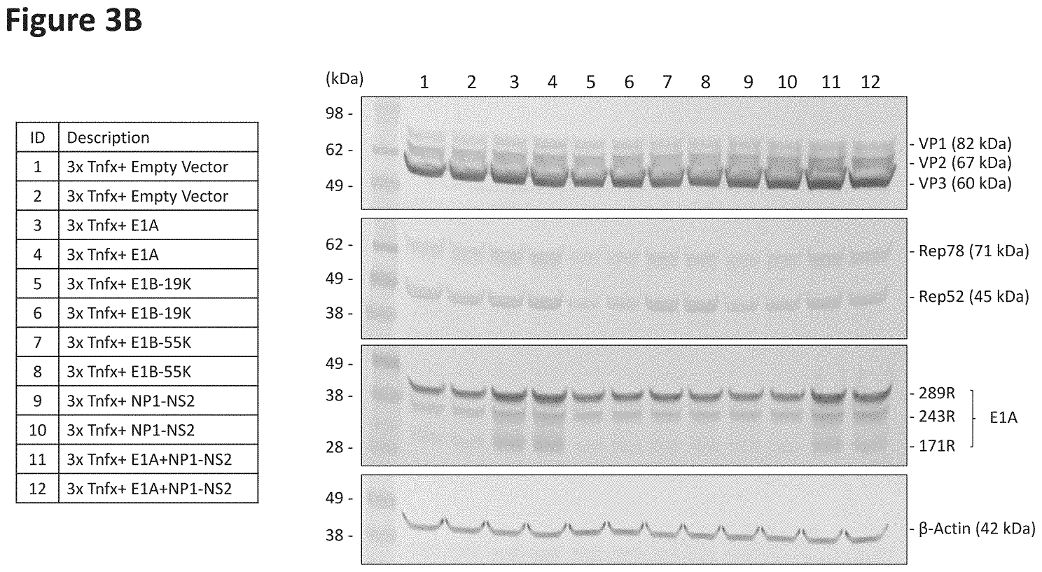

The experiments in Example 1 were repeated with the following conditions: empty vector (control), E1A alone, E1B-19k alone, E1B-55k alone, NP1-NS2, and E1A+NP1-NS2, and the crude virus titer results were analyzed by ddPCR. As shown in FIG. 3A, E1A alone or E1A plus the bocavirus NP1 and NS2 genes increased the AAV titer by about 50%, while bocavirus genes alone had no effect.

To confirm the over-expression of E1A, Western blot analysis was performed to evaluate E1A protein expression (transfection conditions shown in the Table of FIG. 3B). As shown in FIG. 3B, the additional co-transfection of E1A expressed higher levels of all three isoforms of E1A protein (289R, 243R, and 171R) in samples 3, 4, 11, and 12, which had plasmid-transfected E1A, as compared to samples 1 and 2, which only contained E1A proteins endogenously expressed by HEK293 cells. β-actin levels were measured as a control, and no significant differences were found between the different conditions. The protein expression levels of Rep (Rep78 and Rep52 isoforms) and Cap (VP1, VP2, and VP3) were also determined, and no significant difference was detected between the E1A-transfected samples and the controls. Thus, E1A overexpression may improve AAV titer through mechanisms other than regulation of Rep and Cap protein expression.

### Example 3. Dosage-Dependent Improvement by E1A for AAV Production

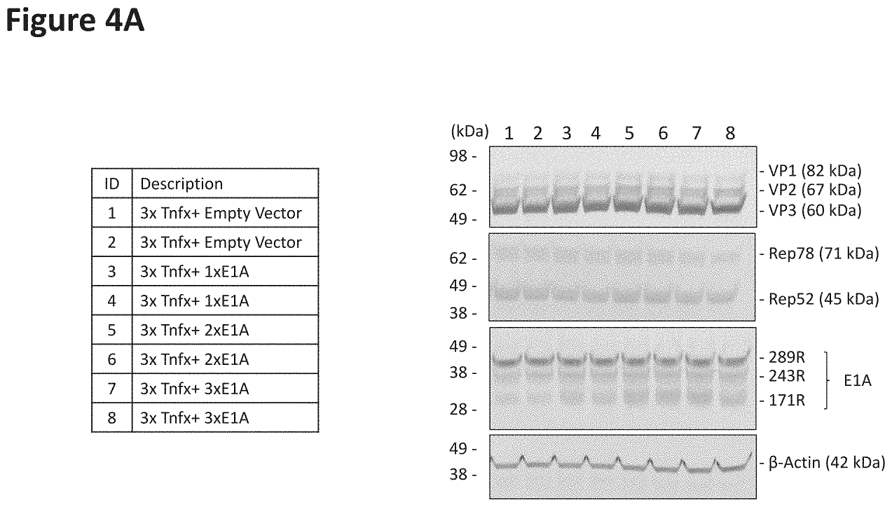

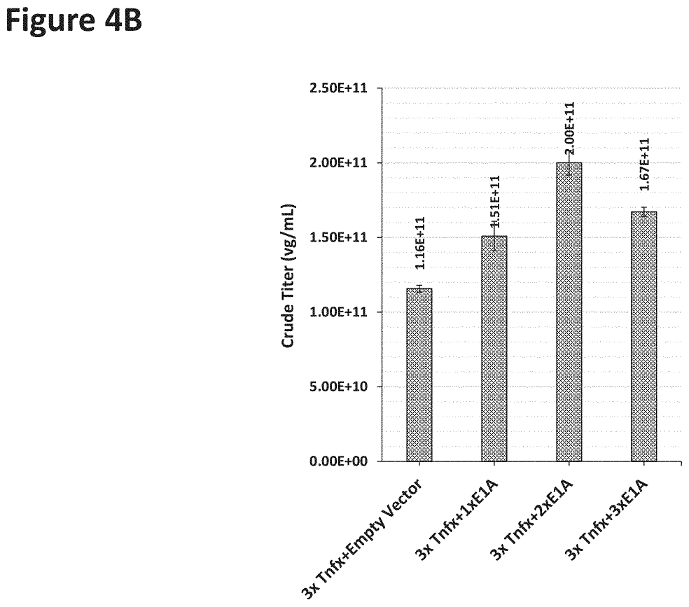

The E1A function for AAV titer improvement was further tested by titration of the E1A plasmids at 1:1, 2:1, or 3:1 molar ratio to the pHelper plasmid, indicated in the Table in FIG. 4A as 1×E1A, 2×E1A, or 3×E1A, respectively. Transfection and AAV production were performed in the same manner as Example 1. The increase of E1A overexpression was confirmed by Western blot analysis, as shown in FIG. 4A. The AAV9.GFP titer increased in an E1A-dosage dependent manner, as shown in FIG. 4B.

### Example 4. Novel and Enhanced Triple Transfection for AAV Production

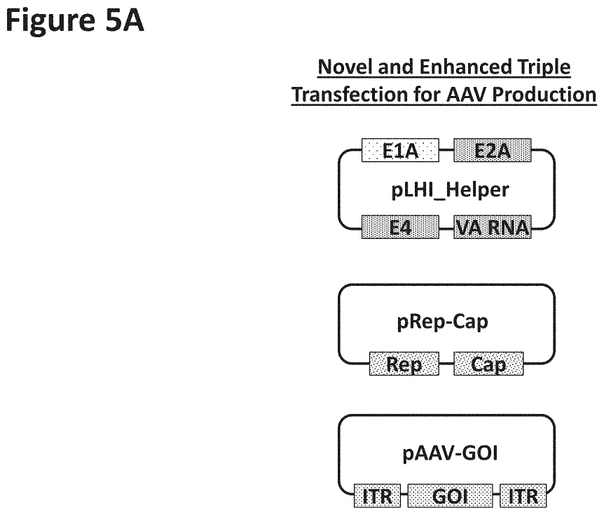

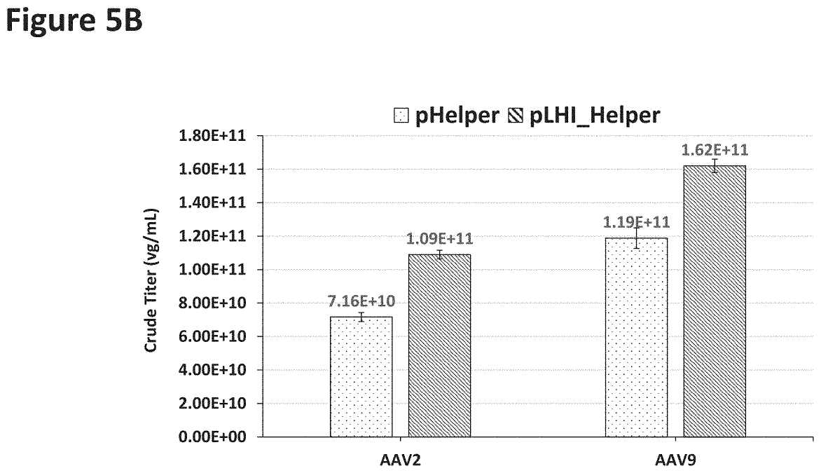

To simplify the transfection experiment, the E1A expression cassette was subcloned from the pcDNA3.1-E1A plasmid of Example 1, into the standard pHelper vector shown in FIG. 1. The new helper plasmid, termed “pLHI_Helper” (SEQ ID NO:5), was transfected into HEK293 cells in lieu of the conventional pHelper as shown in FIG. 5A. Compared to the standard triple transfection, the pLHI_Helper-mediated triple transfection significantly improved the AAV2.GFP and AAV9.GFP production by 52.2% and 36.1%, respectively, over the control standard triple transfection using pHelper, as shown in FIG. 5B.

SEQUENCES

SEQ ID NO: 1-Exemplary plasmid containing E1A (pcDNA3.1-E1A)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTgccaccatgagacatattatctgccacggaggtgttattaccga
agaaatggccgccagtcttttggaccagctgatcgaagaggtactggctgataatcttccacct
cctagccattttgaaccacctacccttcacgaactgtatgatttagacgtgacggcccccgaag
atcccaacgaggaggcggtttcgcagatttttcccgactctgtaatgttggcggtgcaggaagg
gattgacttactcacttttccgccggcgcccggttctccggagccgcctcacctttcccggcag
cccgagcagccggagcagagagccttgggtccggtttctatgccaaaccttgtaccggaggtga
tcgatcttacctgccacgaggctggctttccacccagtgacgacgaggatgaagagggtgagga
gtttgtgttagattatgtggagcaccccgggcacggttgcaggtcttgtcattatcaccggagg
aatacgggggacccagatattatgtgttcgctttgctatatgaggacctgtggcatgtttgtct
acagtaagtgaaaattatgggcagtgggtgatagagtggtgggtttggtgtggtaatttttttt
ttaatttttacagttttgtggtttaaagaattttgtattgtgatttttttaaaaggtcctgtgt
ctgaacctgagcctgagcccgagccagaaccggagcctgcaagacctacccgccgtcctaaaat
ggcgcctgctatcctgagacgcccgacatcacctgtgtctagagaatgcaatagtagtacggat
agctgtgactccggtccttctaacacacctcctgagatacacccggtggtcccgctgtgcccca
ttaaaccagttgccgtgagagttggtgggcgtcgccaggctgtggaatgtatcgaggacttgct
taacgagcctgggcaacctttggacttgagctgtaaacgccccaggccataaGAATTCTGCAGA
TATCCAGCACAGTGGCGGCCGCTCGAGTCTAGAGGGCCCGTTTAAACCCGCTGATCAGCCTCGA
CTGTGCCTTCTAGTTGCCAGCCATCTGTTGTTTGCCCCTCCCCCGTGCCTTCCTTGACCCTGGA
AGGTGCCACTCCCACTGTCCTTTCCTAATAAAATGAGGAAATTGCATCGCATTGTCTGAGTAGG
TGTCATTCTATTCTGGGGGGTGGGGTGGGGCAGGACAGCAAGGGGGAGGATTGGGAAGACAATA
GCAGGCATGCTGGGGATGCGGTGGGCTCTATGGCTTCTGAGGCGGAAAGAACCAGCTGGGGCTC
TAGGGGGTATCCCCACGCGCCCTGTAGCGGCGCATTAAGCGCGGCGGGTGTGGTGGTTACGCGC
AGCGTGACCGCTACACTTGCCAGCGCCCTAGCGCCCGCTCCTTTCGCTTTCTTCCCTTCCTTTC
TCGCCACGTTCGCCGGCTTTCCCCGTCAAGCTCTAAATCGGGGGCTCCCTTTAGGGTTCCGATT
TAGTGCTTTACGGCACCTCGACCCCAAAAAACTTGATTAGGGTGATGGTTCACGTAGTGGGCCA
TCGCCCTGATAGACGGTTTTTCGCCCTTTGACGTTGGAGTCCACGTTCTTTAATAGTGGACTCT
TGTTCCAAACTGGAACAACACTCAACCCTATCTCGGTCTATTCTTTTGATTTATAAGGGATTTT
GCCGATTTCGGCCTATTGGTTAAAAAATGAGCTGATTTAACAAAAATTTAACGCGAATTAATTC
TGTGGAATGTGTGTCAGTTAGGGTGTGGAAAGTCCCCAGGCTCCCCAGCAGGCAGAAGTATGCA
AAGCATGCATCTCAATTAGTCAGCAACCAGGTGTGGAAAGTCCCCAGGCTCCCCAGCAGGCAGA
AGTATGCAAAGCATGCATCTCAATTAGTCAGCAACCATAGTCCCGCCCCTAACTCCGCCCATCC
CGCCCCTAACTCCGCCCAGTTCCGCCCATTCTCCGCCCCATGGCTGACTAATTTTTTTTATTTA
TGCAGAGGCCGAGGCCGCCTCTGCCTCTGAGCTATTCCAGAAGTAGTGAGGAGGCTTTTTTGGA
GGCCTAGGCTTTTGCAAAAAGCTCCCGGGAGCTTGTATATCCATTTTCGGATCTGATCAAGAGA
CAGGATGAGGATCGTTTCGCATGATTGAACAAGATGGATTGCACGCAGGTTCTCCGGCCGCTTG
GGTGGAGAGGCTATTCGGCTATGACTGGGCACAACAGACAATCGGCTGCTCTGATGCCGCCGTG
TTCCGGCTGTCAGCGCAGGGGCGCCCGGTTCTTTTTGTCAAGACCGACCTGTCCGGTGCCCTGA
ATGAACTGCAGGACGAGGCAGCGCGGCTATCGTGGCTGGCCACGACGGGCGTTCCTTGCGCAGC
TGTGCTCGACGTTGTCACTGAAGCGGGAAGGGACTGGCTGCTATTGGGCGAAGTGCCGGGGCAG
GATCTCCTGTCATCTCACCTTGCTCCTGCCGAGAAAGTATCCATCATGGCTGATGCAATGCGGC
GGCTGCATACGCTTGATCCGGCTACCTGCCCATTCGACCACCAAGCGAAACATCGCATCGAGCG
AGCACGTACTCGGATGGAAGCCGGTCTTGTCGATCAGGATGATCTGGACGAAGAGCATCAGGGG
CTCGCGCCAGCCGAACTGTTCGCCAGGCTCAAGGCGCGCATGCCCGACGGCGAGGATCTCGTCG
TGACCCATGGCGATGCCTGCTTGCCGAATATCATGGTGGAAAATGGCCGCTTTTCTGGATTCAT
CGACTGTGGCCGGCTGGGTGTGGCGGACCGCTATCAGGACATAGCGTTGGCTACCCGTGATATT
GCTGAAGAGCTTGGCGGCGAATGGGCTGACCGCTTCCTCGTGCTTTACGGTATCGCCGCTCCCG
ATTCGCAGCGCATCGCCTTCTATCGCCTTCTTGACGAGTTCTTCTGAGCGGGACTCTGGGGTTC
GAAATGACCGACCAAGCGACGCCCAACCTGCCATCACGAGATTTCGATTCCACCGCCGCCTTCT
ATGAAAGGTTGGGCTTCGGAATCGTTTTCCGGGACGCCGGCTGGATGATCCTCCAGCGCGGGGA
TCTCATGCTGGAGTTCTTCGCCCACCCCAACTTGTTTATTGCAGCTTATAATGGTTACAAATAA
AGCAATAGCATCACAAATTTCACAAATAAAGCATTTTTTTCACTGCATTCTAGTTGTGGTTTGT
CCAAACTCATCAATGTATCTTATCATGTCTGTATACCGTCGACCTCTAGCTAGAGCTTGGCGTA
ATCATGGTCATAGCTGTTTCCTGTGTGAAATTGTTATCCGCTCACAATTCCACACAACATACGA
GCCGGAAGCATAAAGTGTAAAGCCTGGGGTGCCTAATGAGTGAGCTAACTCACATTAATTGCGT
TGCGCTCACTGCCCGCTTTCCAGTCGGGAAACCTGTCGTGCCAGCTGCATTAATGAATCGGCCA
ACGCGCGGGGAGAGGCGGTTTGCGTATTGGGCGCTCTTCCGCTTCCTCGCTCACTGACTCGCTG
CGCTCGGTCGTTCGGCTGCGGCGAGCGGTATCAGCTCACTCAAAGGCGGTAATACGGTTATCCA
CAGAATCAGGGGATAACGCAGGAAAGAACATGTGAGCAAAAGGCCAGCAAAAGGCCAGGAACCG
TAAAAAGGCCGCGTTGCTGGCGTTTTTCCATAGGCTCCGCCCCCCTGACGAGCATCACAAAAAT
CGACGCTCAAGTCAGAGGTGGCGAAACCCGACAGGACTATAAAGATACCAGGCGTTTCCCCCTG
GAAGCTCCCTCGTGCGCTCTCCTGTTCCGACCCTGCCGCTTACCGGATACCTGTCCGCCTTTCT
CCCTTCGGGAAGCGTGGCGCTTTCTCATAGCTCACGCTGTAGGTATCTCAGTTCGGTGTAGGTC
GTTCGCTCCAAGCTGGGCTGTGTGCACGAACCCCCCGTTCAGCCCGACCGCTGCGCCTTATCCG
GTAACTATCGTCTTGAGTCCAACCCGGTAAGACACGACTTATCGCCACTGGCAGCAGCCACTGG
TAACAGGATTAGCAGAGCGAGGTATGTAGGCGGTGCTACAGAGTTCTTGAAGTGGTGGCCTAAC
TACGGCTACACTAGAAGAACAGTATTTGGTATCTGCGCTCTGCTGAAGCCAGTTACCTTCGGAA
AAAGAGTTGGTAGCTCTTGATCCGGCAAACAAACCACCGCTGGTAGCGGTTTTTTTGTTTGCAA
GCAGCAGATTACGCGCAGAAAAAAAGGATCTCAAGAAGATCCTTTGATCTTTTCTACGGGGTCT
GACGCTCAGTGGAACGAAAACTCACGTTAAGGGATTTTGGTCATGAGATTATCAAAAAGGATCT
TCACCTAGATCCTTTTAAATTAAAAATGAAGTTTTAAATCAATCTAAAGTATATATGAGTAAAC
TTGGTCTGACAGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGGCTTACCATCTG
GCCCCAGTGCTGCAATGATACCGCGAGACCCACGCTCACCGGCTCCAGATTTATCAGCAATAAA
CCAGCCAGCCGGAAGGGCCGAGCGCAGAAGTGGTCCTGCAACTTTATCCGCCTCCATCCAGTCT
ATTAATTGTTGCCGGGAAGCTAGAGTAAGTAGTTCGCCAGTTAATAGTTTGCGCAACGTTGTTG
CCATTGCTACAGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTAGCTCCTTCGGT
CCTCCGATCGTTGTCAGAAGTAAGTTGGCCGCAGTGTTATCACTCATGGTTATGGCAGCACTGC
ATAATTCTCTTACTGTCATGCCATCCGTAAGATGCTTTTCTGTGACTGGTGAGTACTCAACCAA
GTCATTCTGAGAATAGTGTATGCGGCGACCGAGTTGCTCTTGCCCGGCGTCAATACGGGATAAT
ACCGCGCCACATAGCAGAACTTTAAAAGTGCTCATCATTGGAAAACGTTCTTCGGGGCGAAAAC
TCTCAAGGATCTTACCGCTGTTGAGATCCAGTTCGATGTAACCCACTCGTGCACCCAACTGATC
TTCAGCATCTTTTACTTTCACCAGCGTTTCTGGGTGAGCAAAAACAGGAAGGCAAAATGCCGCA
AAAAAGGGAATAAGGGCGACACGGAAATGTTGAATACTCATACTCTTCCTTTTTCAATATTATT
GAAGCATTTATCAGGGTTATTGTCTCATGAGCGGATACATATTTGAATGTATTTAGAAAAATAA
ACAAATAGGGGTTCCGCGCACATTTCCCCGAAAAGTGCCACCTGACGTC
SEQ ID NO: 2-Exemplary plasmid containing E1B-19k (pcDNA3.1-E1B-19K)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTttgcagaagttggtcgtgaggcactgggcaggtaagtatcaagg
ttacaagacaggtttaaggagaccaatagaaactgggcttgtcgagacagagaagactcttgcg
tttctgataggcacctattggtcttactgacatccactttgcctttctctccacaggtgtccac
tcccagttcaattacagctcttGCCACCatggaggcttgggagtgtttggaagatttttctgct
gtgcgtaacttgctggaacagagctctaacagtacctcttggttttggaggtttctgtggggct
catcccaggcaaagttagtctgcagaattaaggaggattacaagtgggaatttgaagagctttt
gaaatcctgtggtgagctgtttgattctttgaatctgggtcaccaggcgcttttccaagagaag
gtcatcaagactttggatttttccacaccggggcgcgctgcggctgctgttgcttttttgagtt
ttataaaggataaatggagcgaagaaacccatctgagcggggggtacctgctggattttctggc
catgcatctgtggagagcggttgtgagacacaagaatcgcctgctactgttgtcttccgtccgc
ccggcgataataccgacggaggagcagcagcagcagcaggaggaagccaggcggcggcggcagg
agcagagcccatggaacccgagagccggcctggaccctcgggaatgaGAATTCTGCAGATATCC
AGCACAGTGGCGGCCGCTCGAGTCTAGAGGGCCCGTTTAAACCCGCTGATCAGCCTCGACTGTG
CCTTCTAGTTGCCAGCCATCTGTTGTTTGCCCCTCCCCCGTGCCTTCCTTGACCCTGGAAGGTG
CCACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAGCAGG
CATGCTGGGGATGCGGTGGGCTCTATGGCTTCTGAGGCGGAAAGAACCAGCTGGGGCTCTAGGG
GGTATCCCCACGCGCCCTGTAGCGGCGCATTAAGCGCGGCGGGTGTGGTGGTTACGCGCAGCGT
GACCGCTACACTTGCCAGCGCCCTAGCGCCCGCTCCTTTCGCTTTCTTCCCTTCCTTTCTCGCC
ACGTTCGCCGGCTTTCCCCGTCAAGCTCTAAATCGGGGGCTCCCTTTAGGGTTCCGATTTAGTG
CTTTACGGCACCTCGACCCCAAAAAACTTGATTAGGGTGATGGTTCACGTAGTGGGCCATCGCC
CTGATAGACGGTTTTTCGCCCTTTGACGTTGGAGTCCACGTTCTTTAATAGTGGACTCTTGTTC
CAAACTGGAACAACACTCAACCCTATCTCGGTCTATTCTTTTGATTTATAAGGGATTTTGCCGA
TTTCGGCCTATTGGTTAAAAAATGAGCTGATTTAACAAAAATTTAACGCGAATTAATTCTGTGG
AATGTGTGTCAGTTAGGGTGTGGAAAGTCCCCAGGCTCCCCAGCAGGCAGAAGTATGCAAAGCA
TGCATCTCAATTAGTCAGCAACCAGGTGTGGAAAGTCCCCAGGCTCCCCAGCAGGCAGAAGTAT
GCAAAGCATGCATCTCAATTAGTCAGCAACCATAGTCCCGCCCCTAACTCCGCCCATCCCGCCC
CTAACTCCGCCCAGTTCCGCCCATTCTCCGCCCCATGGCTGACTAATTTTTTTTATTTATGCAG
AGGCCGAGGCCGCCTCTGCCTCTGAGCTATTCCAGAAGTAGTGAGGAGGCTTTTTTGGAGGCCT
AGGCTTTTGCAAAAAGCTCCCGGGAGCTTGTATATCCATTTTCGGATCTGATCAAGAGACAGGA
TGAGGATCGTTTCGCATGATTGAACAAGATGGATTGCACGCAGGTTCTCCGGCCGCTTGGGTGG
AGAGGCTATTCGGCTATGACTGGGCACAACAGACAATCGGCTGCTCTGATGCCGCCGTGTTCCG
GCTGTCAGCGCAGGGGCGCCCGGTTCTTTTTGTCAAGACCGACCTGTCCGGTGCCCTGAATGAA
CTGCAGGACGAGGCAGCGCGGCTATCGTGGCTGGCCACGACGGGCGTTCCTTGCGCAGCTGTGC
TCGACGTTGTCACTGAAGCGGGAAGGGACTGGCTGCTATTGGGCGAAGTGCCGGGGCAGGATCT
CCTGTCATCTCACCTTGCTCCTGCCGAGAAAGTATCCATCATGGCTGATGCAATGCGGCGGCTG
CATACGCTTGATCCGGCTACCTGCCCATTCGACCACCAAGCGAAACATCGCATCGAGCGAGCAC
GTACTCGGATGGAAGCCGGTCTTGTCGATCAGGATGATCTGGACGAAGAGCATCAGGGGCTCGC
GCCAGCCGAACTGTTCGCCAGGCTCAAGGCGCGCATGCCCGACGGCGAGGATCTCGTCGTGACC
CATGGCGATGCCTGCTTGCCGAATATCATGGTGGAAAATGGCCGCTTTTCTGGATTCATCGACT
GTGGCCGGCTGGGTGTGGCGGACCGCTATCAGGACATAGCGTTGGCTACCCGTGATATTGCTGA
AGAGCTTGGCGGCGAATGGGCTGACCGCTTCCTCGTGCTTTACGGTATCGCCGCTCCCGATTCG
CAGCGCATCGCCTTCTATCGCCTTCTTGACGAGTTCTTCTGAGCGGGACTCTGGGGTTCGAAAT
GACCGACCAAGCGACGCCCAACCTGCCATCACGAGATTTCGATTCCACCGCCGCCTTCTATGAA
AGGTTGGGCTTCGGAATCGTTTTCCGGGACGCCGGCTGGATGATCCTCCAGCGCGGGGATCTCA
TGCTGGAGTTCTTCGCCCACCCCAACTTGTTTATTGCAGCTTATAATGGTTACAAATAAAGCAA
TAGCATCACAAATTTCACAAATAAAGCATTTTTTTCACTGCATTCTAGTTGTGGTTTGTCCAAA
CTCATCAATGTATCTTATCATGTCTGTATACCGTCGACCTCTAGCTAGAGCTTGGCGTAATCAT
GGTCATAGCTGTTTCCTGTGTGAAATTGTTATCCGCTCACAATTCCACACAACATACGAGCCGG
AAGCATAAAGTGTAAAGCCTGGGGTGCCTAATGAGTGAGCTAACTCACATTAATTGCGTTGCGC
TCACTGCCCGCTTTCCAGTCGGGAAACCTGTCGTGCCAGCTGCATTAATGAATCGGCCAACGCG
CGGGGAGAGGCGGTTTGCGTATTGGGCGCTCTTCCGCTTCCTCGCTCACTGACTCGCTGCGCTC
GGTCGTTCGGCTGCGGCGAGCGGTATCAGCTCACTCAAAGGCGGTAATACGGTTATCCACAGAA
TCAGGGGATAACGCAGGAAAGAACATGTGAGCAAAAGGCCAGCAAAAGGCCAGGAACCGTAAAA
AGGCCGCGTTGCTGGCGTTTTTCCATAGGCTCCGCCCCCCTGACGAGCATCACAAAAATCGACG
CTCAAGTCAGAGGTGGCGAAACCCGACAGGACTATAAAGATACCAGGCGTTTCCCCCTGGAAGC
TCCCTCGTGCGCTCTCCTGTTCCGACCCTGCCGCTTACCGGATACCTGTCCGCCTTTCTCCCTT
CGGGAAGCGTGGCGCTTTCTCATAGCTCACGCTGTAGGTATCTCAGTTCGGTGTAGGTCGTTCG
CTCCAAGCTGGGCTGTGTGCACGAACCCCCCGTTCAGCCCGACCGCTGCGCCTTATCCGGTAAC
TATCGTCTTGAGTCCAACCCGGTAAGACACGACTTATCGCCACTGGCAGCAGCCACTGGTAACA
GGATTAGCAGAGCGAGGTATGTAGGCGGTGCTACAGAGTTCTTGAAGTGGTGGCCTAACTACGG
CTACACTAGAAGAACAGTATTTGGTATCTGCGCTCTGCTGAAGCCAGTTACCTTCGGAAAAAGA
GTTGGTAGCTCTTGATCCGGCAAACAAACCACCGCTGGTAGCGGTTTTTTTGTTTGCAAGCAGC
AGATTACGCGCAGAAAAAAAGGATCTCAAGAAGATCCTTTGATCTTTTCTACGGGGTCTGACGC
TCAGTGGAACGAAAACTCACGTTAAGGGATTTTGGTCATGAGATTATCAAAAAGGATCTTCACC
TAGATCCTTTTAAATTAAAAATGAAGTTTTAAATCAATCTAAAGTATATATGAGTAAACTTGGT
CTGACAGTTACCAATGCTTAATCAGTGAGGCACCTATCTCAGCGATCTGTCTATTTCGTTCATC
CATAGTTGCCTGACTCCCCGTCGTGTAGATAACTACGATACGGGAGGGCTTACCATCTGGCCCC
AGTGCTGCAATGATACCGCGAGACCCACGCTCACCGGCTCCAGATTTATCAGCAATAAACCAGC
CAGCCGGAAGGGCCGAGCGCAGAAGTGGTCCTGCAACTTTATCCGCCTCCATCCAGTCTATTAA
TTGTTGCCGGGAAGCTAGAGTAAGTAGTTCGCCAGTTAATAGTTTGCGCAACGTTGTTGCCATT
GCTACAGGCATCGTGGTGTCACGCTCGTCGTTTGGTATGGCTTCATTCAGCTCCGGTTCCCAAC
GATCAAGGCGAGTTACATGATCCCCCATGTTGTGCAAAAAAGCGGTTAGCTCCTTCGGTCCTCC
GATCGTTGTCAGAAGTAAGTTGGCCGCAGTGTTATCACTCATGGTTATGGCAGCACTGCATAAT
TCTCTTACTGTCATGCCATCCGTAAGATGCTTTTCTGTGACTGGTGAGTACTCAACCAAGTCAT
TCTGAGAATAGTGTATGCGGCGACCGAGTTGCTCTTGCCCGGCGTCAATACGGGATAATACCGC
GCCACATAGCAGAACTTTAAAAGTGCTCATCATTGGAAAACGTTCTTCGGGGCGAAAACTCTCA
AGGATCTTACCGCTGTTGAGATCCAGTTCGATGTAACCCACTCGTGCACCCAACTGATCTTCAG
CATCTTTTACTTTCACCAGCGTTTCTGGGTGAGCAAAAACAGGAAGGCAAAATGCCGCAAAAAA
GGGAATAAGGGCGACACGGAAATGTTGAATACTCATACTCTTCCTTTTTCAATATTATTGAAGC
ATTTATCAGGGTTATTGTCTCATGAGCGGATACATATTTGAATGTATTTAGAAAAATAAACAAA
TAGGGGTTCCGCGCACATTTCCCCGAAAAGTGCCACCTGACGTC
SEQ ID NO: 3-Exemplary plasmid containing E1B-55k (pcDNA3.1-E1B-55K)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGAGCTCGGATCCttgcagaagttggtcgtgaggcactg
ggcaggtaagtatcaaggttacaagacaggtttaaggagaccaatagaaactgggcttgtcgag
acagagaagactcttgcgtttctgataggcacctattggtcttactgacatccactttgccttt
ctctccacaggtgtccactcccagttcaattacagctcttGCCACCatggagcgaagaaaccca
tctgagcggggggtacctgctggattttctggccatgcatctgtggagagcggttgtgagacac
aagaatcgcctgctactgttgtcttccgtccgcccggcgataataccgacggaggagcagcagc
agcagcaggaggaagccaggcggcggcggcaggagcagagcccatggaacccgagagccggcct
ggaccctcgggaatgaatgttgtacaggtggctgaactgtatccagaactgagacgcattttga
caattacagaggatgggcaggggctaaagggggtaaagagggagcggggggcttgtgaggctac
agaggaggctaggaatctagcttttagcttaatgaccagacaccgtcctgagtgtattactttt
caacagatcaaggataattgcgctaatgagcttgatctgctggcgcagaagtattccatagagc
agctgaccacttactggctgcagccaggggatgattttgaggaggctattagggtatatgcaaa
ggtggcacttaggccagattgcaagtacaagatcagcaaacttgtaaatatcaggaattgttgc
tacatttctgggaacggggccgaggtggagatagatacggaggatagggtggcctttagatgta
gcatgataaatatgtggccgggggtgcttggcatggacggggtggttattatgaatgtaaggtt
tactggccccaattttagcggtacggttttcctggccaataccaaccttatcctacacggtgta
agcttctatgggtttaacaatacctgtgtggaagcctggaccgatgtaagggttcggggctgtg
ccttttactgctgctggaagggggtggtgtgtcgccccaaaagcagggcttcaattaagaaatg
cctctttgaaaggtgtaccttgggtatcctgtctgagggtaactccagggtgcgccacaatgtg
gcctccgactgtggttgcttcatgctagtgaaaagcgtggctgtgattaagcataacatggtat
gtggcaactgcgaggacagggcctctcagatgctgacctgctcggacggcaactgtcacctgct
gaagaccattcacgtagccagccactctcgcaaggcctggccagtgtttgagcataacatactg
acccgctgttccttgcatttgggtaacaggaggggggtgttcctaccttaccaatgcaatttga
gtcacactaagatattgcttgagcccgagagcatgtccaaggtgaacctgaacggggtgtttga
catgaccatgaagatctggaaggtgctgaggtacgatgagacccgcaccaggtgcagaccctgc
gagtgtggcggtaaacatattaggaaccagcctgtgatgctggatgtgaccgaggagctgaggc
ccgatcacttggtgctggcctgcacccgcgctgagtttggctctagcgatgaagatacagattg
aGAATTCTGCAGATATCCAGCACAGTGGCGGCCGCTCGAGTCTAGAGGGCCCGTTTAAACCCGC
TGATCAGCCTCGACTGTGCCTTCTAGTTGCCAGCCATCTGTTGTTTGCCCCTCCCCCGTGCCTT
CCTTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGGAGGAT
TGGGAAGACAATAGCAGGCATGCTGGGGATGCGGTGGGCTCTATGGCTTCTGAGGCGGAAAGAA
CCAGCTGGGGCTCTAGGGGGTATCCCCACGCGCCCTGTAGCGGCGCATTAAGCGCGGCGGGTGT
GGTGGTTACGCGCAGCGTGACCGCTACACTTGCCAGCGCCCTAGCGCCCGCTCCTTTCGCTTTC
TTCCCTTCCTTTCTCGCCACGTTCGCCGGCTTTCCCCGTCAAGCTCTAAATCGGGGGCTCCCTT
TAGGGTTCCGATTTAGTGCTTTACGGCACCTCGACCCCAAAAAACTTGATTAGGGTGATGGTTC
ACGTAGTGGGCCATCGCCCTGATAGACGGTTTTTCGCCCTTTGACGTTGGAGTCCACGTTCTTT
AATAGTGGACTCTTGTTCCAAACTGGAACAACACTCAACCCTATCTCGGTCTATTCTTTTGATT
TATAAGGGATTTTGCCGATTTCGGCCTATTGGTTAAAAAATGAGCTGATTTAACAAAAATTTAA
CGCGAATTAATTCTGTGGAATGTGTGTCAGTTAGGGTGTGGAAAGTCCCCAGGCTCCCCAGCAG
GCAGAAGTATGCAAAGCATGCATCTCAATTAGTCAGCAACCAGGTGTGGAAAGTCCCCAGGCTC
CCCAGCAGGCAGAAGTATGCAAAGCATGCATCTCAATTAGTCAGCAACCATAGTCCCGCCCCTA
ACTCCGCCCATCCCGCCCCTAACTCCGCCCAGTTCCGCCCATTCTCCGCCCCATGGCTGACTAA
TTTTTTTTATTTATGCAGAGGCCGAGGCCGCCTCTGCCTCTGAGCTATTCCAGAAGTAGTGAGG
AGGCTTTTTTGGAGGCCTAGGCTTTTGCAAAAAGCTCCCGGGAGCTTGTATATCCATTTTCGGA
TCTGATCAAGAGACAGGATGAGGATCGTTTCGCATGATTGAACAAGATGGATTGCACGCAGGTT
CTCCGGCCGCTTGGGTGGAGAGGCTATTCGGCTATGACTGGGCACAACAGACAATCGGCTGCTC
TGATGCCGCCGTGTTCCGGCTGTCAGCGCAGGGGCGCCCGGTTCTTTTTGTCAAGACCGACCTG
TCCGGTGCCCTGAATGAACTGCAGGACGAGGCAGCGCGGCTATCGTGGCTGGCCACGACGGGCG
TTCCTTGCGCAGCTGTGCTCGACGTTGTCACTGAAGCGGGAAGGGACTGGCTGCTATTGGGCGA
AGTGCCGGGGCAGGATCTCCTGTCATCTCACCTTGCTCCTGCCGAGAAAGTATCCATCATGGCT
GATGCAATGCGGCGGCTGCATACGCTTGATCCGGCTACCTGCCCATTCGACCACCAAGCGAAAC
ATCGCATCGAGCGAGCACGTACTCGGATGGAAGCCGGTCTTGTCGATCAGGATGATCTGGACGA
AGAGCATCAGGGGCTCGCGCCAGCCGAACTGTTCGCCAGGCTCAAGGCGCGCATGCCCGACGGC
GAGGATCTCGTCGTGACCCATGGCGATGCCTGCTTGCCGAATATCATGGTGGAAAATGGCCGCT
TTTCTGGATTCATCGACTGTGGCCGGCTGGGTGTGGCGGACCGCTATCAGGACATAGCGTTGGC
TACCCGTGATATTGCTGAAGAGCTTGGCGGCGAATGGGCTGACCGCTTCCTCGTGCTTTACGGT
ATCGCCGCTCCCGATTCGCAGCGCATCGCCTTCTATCGCCTTCTTGACGAGTTCTTCTGAGCGG
GACTCTGGGGTTCGAAATGACCGACCAAGCGACGCCCAACCTGCCATCACGAGATTTCGATTCC
ACCGCCGCCTTCTATGAAAGGTTGGGCTTCGGAATCGTTTTCCGGGACGCCGGCTGGATGATCC
TCCAGCGCGGGGATCTCATGCTGGAGTTCTTCGCCCACCCCAACTTGTTTATTGCAGCTTATAA
TGGTTACAAATAAAGCAATAGCATCACAAATTTCACAAATAAAGCATTTTTTTCACTGCATTCT
AGTTGTGGTTTGTCCAAACTCATCAATGTATCTTATCATGTCTGTATACCGTCGACCTCTAGCT
AGAGCTTGGCGTAATCATGGTCATAGCTGTTTCCTGTGTGAAATTGTTATCCGCTCACAATTCC
ACACAACATACGAGCCGGAAGCATAAAGTGTAAAGCCTGGGGTGCCTAATGAGTGAGCTAACTC
ACATTAATTGCGTTGCGCTCACTGCCCGCTTTCCAGTCGGGAAACCTGTCGTGCCAGCTGCATT
AATGAATCGGCCAACGCGCGGGGAGAGGCGGTTTGCGTATTGGGCGCTCTTCCGCTTCCTCGCT
CACTGACTCGCTGCGCTCGGTCGTTCGGCTGCGGCGAGCGGTATCAGCTCACTCAAAGGCGGTA
ATACGGTTATCCACAGAATCAGGGGATAACGCAGGAAAGAACATGTGAGCAAAAGGCCAGCAAA
AGGCCAGGAACCGTAAAAAGGCCGCGTTGCTGGCGTTTTTCCATAGGCTCCGCCCCCCTGACGA
GCATCACAAAAATCGACGCTCAAGTCAGAGGTGGCGAAACCCGACAGGACTATAAAGATACCAG
GCGTTTCCCCCTGGAAGCTCCCTCGTGCGCTCTCCTGTTCCGACCCTGCCGCTTACCGGATACC
TGTCCGCCTTTCTCCCTTCGGGAAGCGTGGCGCTTTCTCATAGCTCACGCTGTAGGTATCTCAG
TTCGGTGTAGGTCGTTCGCTCCAAGCTGGGCTGTGTGCACGAACCCCCCGTTCAGCCCGACCGC
TGCGCCTTATCCGGTAACTATCGTCTTGAGTCCAACCCGGTAAGACACGACTTATCGCCACTGG
CAGCAGCCACTGGTAACAGGATTAGCAGAGCGAGGTATGTAGGCGGTGCTACAGAGTTCTTGAA
GTGGTGGCCTAACTACGGCTACACTAGAAGAACAGTATTTGGTATCTGCGCTCTGCTGAAGCCA
GTTACCTTCGGAAAAAGAGTTGGTAGCTCTTGATCCGGCAAACAAACCACCGCTGGTAGCGGTT
TTTTTGTTTGCAAGCAGCAGATTACGCGCAGAAAAAAAGGATCTCAAGAAGATCCTTTGATCTT
TTCTACGGGGTCTGACGCTCAGTGGAACGAAAACTCACGTTAAGGGATTTTGGTCATGAGATTA
TCAAAAAGGATCTTCACCTAGATCCTTTTAAATTAAAAATGAAGTTTTAAATCAATCTAAAGTA
TATATGAGTAAACTTGGTCTGACAGTTACCAATGCTTAATCAGTGAGGCACCTATCTCAGCGAT
CTGTCTATTTCGTTCATCCATAGTTGCCTGACTCCCCGTCGTGTAGATAACTACGATACGGGAG
GGCTTACCATCTGGCCCCAGTGCTGCAATGATACCGCGAGACCCACGCTCACCGGCTCCAGATT
TATCAGCAATAAACCAGCCAGCCGGAAGGGCCGAGCGCAGAAGTGGTCCTGCAACTTTATCCGC
CTCCATCCAGTCTATTAATTGTTGCCGGGAAGCTAGAGTAAGTAGTTCGCCAGTTAATAGTTTG
CGCAACGTTGTTGCCATTGCTACAGGCATCGTGGTGTCACGCTCGTCGTTTGGTATGGCTTCAT
TCAGCTCCGGTTCCCAACGATCAAGGCGAGTTACATGATCCCCCATGTTGTGCAAAAAAGCGGT
TAGCTCCTTCGGTCCTCCGATCGTTGTCAGAAGTAAGTTGGCCGCAGTGTTATCACTCATGGTT
ATGGCAGCACTGCATAATTCTCTTACTGTCATGCCATCCGTAAGATGCTTTTCTGTGACTGGTG
AGTACTCAACCAAGTCATTCTGAGAATAGTGTATGCGGCGACCGAGTTGCTCTTGCCCGGCGTC
AATACGGGATAATACCGCGCCACATAGCAGAACTTTAAAAGTGCTCATCATTGGAAAACGTTCT
TCGGGGCGAAAACTCTCAAGGATCTTACCGCTGTTGAGATCCAGTTCGATGTAACCCACTCGTG
CACCCAACTGATCTTCAGCATCTTTTACTTTCACCAGCGTTTCTGGGTGAGCAAAAACAGGAAG
GCAAAATGCCGCAAAAAAGGGAATAAGGGCGACACGGAAATGTTGAATACTCATACTCTTCCTT
TTTCAATATTATTGAAGCATTTATCAGGGTTATTGTCTCATGAGCGGATACATATTTGAATGTA
TTTAGAAAAATAAACAAATAGGGGTTCCGCGCACATTTCCCCGAAAAGTGCCACCTGACGTC
SEQ ID NO: 4-Exemplary plasmid containing NP1 and N52 (pcDNA3.1-NP1-N52)
GACGGATCGGGAGATCTCCCGATCCCCTATGGTGCACTCTCAGTACAATCTGCTCTGATGCCGC
ATAGTTAAGCCAGTATCTGCTCCCTGCTTGTGTGTTGGAGGTCGCTGAGTAGTGCGCGAGCAAA
ATTTAAGCTACAACAAGGCAAGGCTTGACCGACAATTGCATGAAGAATCTGCTTAGGGTTAGGC
GTTTTGCGCTGCTTCGCGATGTACGGGCCAGATATACGCGTTGACATTGATTATTGACTAGTTA
TTAATAGTAATCAATTACGGGGTCATTAGTTCATAGCCCATATATGGAGTTCCGCGTTACATAA
CTTACGGTAAATGGCCCGCCTGGCTGACCGCCCAACGACCCCCGCCCATTGACGTCAATAATGA
CGTATGTTCCCATAGTAACGCCAATAGGGACTTTCCATTGACGTCAATGGGTGGAGTATTTACG
GTAAACTGCCCACTTGGCAGTACATCAAGTGTATCATATGCCAAGTACGCCCCCTATTGACGTC
AATGACGGTAAATGGCCCGCCTGGCATTATGCCCAGTACATGACCTTATGGGACTTTCCTACTT
GGCAGTACATCTACGTATTAGTCATCGCTATTACCATGGTGATGCGGTTTTGGCAGTACATCAA
TGGGCGTGGATAGCGGTTTGACTCACGGGGATTTCCAAGTCTCCACCCCATTGACGTCAATGGG
AGTTTGTTTTGGCACCAAAATCAACGGGACTTTCCAAAATGTCGTAACAACTCCGCCCCATTGA
CGCAAATGGGCGGTAGGCGTGTACGGTGGGAGGTCTATATAAGCAGAGCTCTCTGGCTAACTAG
AGAACCCACTGCTTACTGGCTTATCGAAATTAATACGACTCACTATAGGGAGACCCAAGCTGGC
TAGCGTTTAAACTTAAGCTTGGTACCGAGCTCGGATCCACTAGTCCAGTGTGGTGGAATTCttg
cagaagttggtcgtgaggcactgggcaggtaagtatcaaggttacaagacaggtttaaggagac
caatagaaactgggcttgtcgagacagagaagactcttgcgtttctgataggcacctattggtc
ttactgacatccactttgcctttctctccacaggtgtccactcccagttcaattacagctcttG
CCACCATGAGCTCCGGCAACATGAAAGATAAGCACAGAAGCTACAAGAGAAAGGGCTCTCCAGA
GAGGGGCGAGCGCAAGCGGCACTGGCAGACAACCCACCACAGATCTAGATCCAGAAGCCCCATT
AGACATTCTGGAGAGCGGGGAAGCGGCAGCTATCACCAGGAGCACCCTATCAGCCACCTGAGCA
GCTGCACCGCCTCTAAGACCAGCGACCAAGTGATGAAGACAAGAGAATCTACAAGCGGCAAGAA
AGACAACAGAACAAACCCCTACACAGTGTTCAGCCAGCACCGGGCCAGCAATCCTGAGGCTCCT
GGCTGGTGCGGCTTCTACTGGCACAGCACCAGAATCGCCAGAGATGGCACCAACAGCATCTTCA
ACGAGATGAAGCAGCAATTTCAGCAGCTGCAGATCGACAACAAGATCGGCTGGGATAACACCCG
GGAACTGCTGTTTAACCAGAAAAAGACCCTGGACCAGAAGTACAGAAATATGTTCTGGCATTTC
AGAAACAACAGCGACTGTGAACGGTGCAACTACTGGGACGACGTGTACCGGAGACACCTGGCCA
ACGTCTCCTCCCAGACCGAGGCCGATGAAATCACCGACGAGGAAATGCTGAGCGCCGCCGAGAG
CATGGAAGCTGACGCCTCTAATTGAgcgtacagcggctcccgggagttctagggatctgcccct
ctccctcccccccccctaacgttactggccgaagccgcttggaataaggccggtgtgcgtttgt
ctatatgttattttccaccatattgccgtcttttggcaatgtgagggcccggaaacctggccct
gtcttcttgacgagcattcctaggggtctttcccctctcgccaaaggaatgcaaggtctgttga
atgtcgtgaaggaagcagttcctctggaagcttcttgaagacaaacaacgtctgtagcgaccct
ttgcaggcagcggaaccccccacctggcgacaggtgcctctgcggccaaaagccacgtgtataa
gatacacctgcaaaggcggcacaaccccagtgccacgttgtgagttggatagttgtggaaagag
tcaaatggctctcctcaagcgtattcaacaaggggctgaaggatgcccagaaggtaccccattg
tatgggatctgatctggggcctcggtgcacatgctttacatgtgtttagtcgaggttaaaaaaa
cgtctaggccccccgaaccacggggacgtggttttcctttgaaaaacacgatgataaggatcca
ccggagGCCACCATGGCCTTCAACCCCCCCGTGATCCGGGCCTTTAGCCAGCCCGCTTTTACAT
ACGTGTTCAAGTTCCCCTACCCCCAGTGGAAGGAAAAGGAATGGCTGCTGCACGCCCTGCTGGC
CCACGGCACCGAGCAGTCCATGATCCAGCTGCGGAACTGCGCCCCACACCCCGATGAAGACATC
ATTAGAGACGACCTTCTGATCAGCCTGGAAGATAGACACTTCGGCGCCGTTCTGTGCAAAGCCG
TGTACATGGCCACCACCACCCTCATGAGCCACAAGCAGAGAAATATGTTCCCCCGGTGCGACAT
CATCGTCCAGAGCGAGCTGGGAGAAAAGAATCTCCATTGTCACATCATTGTGGGCGGCGAGGGC
CTGTCCAAGAGAAACGCCAAGTCCTCTTGCGCCCAGTTCTACGGCCTGATCCTGGCTGAGATCA
TCCAGAGGTGTAAAAGCCTGCTGGCCACCAGACCATTCGAACCTGAGGAAGCCGACATCTTCCA
CACCCTGAAGAAGGCCGAGCGGGAGGCCTGGGGAGGCGTGACAGGCGGCAACATGCAAATCCTG
CAATACAGAGATAGAAGAGGCGACCTGCATGCACAAACAGTGGATCCTCTGAGATTCTTCAAGA
ATTACCTGCTGCCTAAGAACAGATGCATCAGCTCTTACAGCAAGCCCGATGTGTGCACCTCTCC
TGACAACTGGTTCATCCTGGCCGAGAAGACCTACAGCCACACACTGATCAACGGCCTGCCCCTG
CCTGAACACTACCGGAAGAACTACCACGCCACCCTGGACAACGAAGTGATCCCCGGCCCTCAGA
CCATGGCTTATGGAGGCCGGGGACCATGGGAGCACCTGCCAGAGGACTTTACCCTGCACGAGAA
CGGATACTGCACAGACTGTGGCGGATATCTGCCTCACAGCGCCGACAACAGCATGTACACCGAC
AGAGCCAGCGAGACATCTACCGGCGACATCACACCTAGCGACCTGGGCGATAGCGACGGCGAGG
ACACCGAGCCTGAGACAAGCCAGGTGGACTACTGTCCTCCTAAGAAAAGACGCCTGACCGCCCC
TGCTAGCCCTCCAAACAGCCCTGCCAGCTCTGTGTCCACCATCACATTCTTTAATACCTGGCAC
GCCCAGCCTAGAGATGAGGATGAGCTGAGAGAGTACGAGCGGCAGGCCAGCCTTCTGCAGAAAA
AGCGGGAGAGCAGAAAGCGCGGCGAAGAGGAAACACTGGCTGACAACAGCTCCCAGGAGCAGGA
GCCTCAGCCTGATCCTACCCAGTGGGGCGAAAGACTGGGCTTCATCAGCTCTGGCACGCCCAAC
CAGCCCCCTATCGTGCTGCACTGCTTCGAAGATCTGAGACCTTCTGACGAGGACGAAGGTGAAT
ACATCGGCGAAAAAAGACAGtagCTCGAGTCTAGAGGGCCCGTTTAAACCCGCTGATCAGCCTC
GACTGTGCCTTCTAGTTGCCAGCCATCTGTTGTTTGCCCCTCCCCCGTGCCTTCCTTGACCCTG
GAAGGTGCCACTCCCACTGTCCTTTCCTAATAAAATGAGGAAATTGCATCGCATTGTCTGAGTA
GGTGTCATTCTATTCTGGGGGGTGGGGTGGGGCAGGACAGCAAGGGGGAGGATTGGGAAGACAA
TAGCAGGCATGCTGGGGATGCGGTGGGCTCTATGGCTTCTGAGGCGGAAAGAACCAGCTGGGGC
TCTAGGGGGTATCCCCACGCGCCCTGTAGCGGCGCATTAAGCGCGGCGGGTGTGGTGGTTACGC
GCAGCGTGACCGCTACACTTGCCAGCGCCCTAGCGCCCGCTCCTTTCGCTTTCTTCCCTTCCTT
TCTCGCCACGTTCGCCGGCTTTCCCCGTCAAGCTCTAAATCGGGGGCTCCCTTTAGGGTTCCGA
TTTAGTGCTTTACGGCACCTCGACCCCAAAAAACTTGATTAGGGTGATGGTTCACGTAGTGGGC
CATCGCCCTGATAGACGGTTTTTCGCCCTTTGACGTTGGAGTCCACGTTCTTTAATAGTGGACT
CTTGTTCCAAACTGGAACAACACTCAACCCTATCTCGGTCTATTCTTTTGATTTATAAGGGATT
TTGCCGATTTCGGCCTATTGGTTAAAAAATGAGCTGATTTAACAAAAATTTAACGCGAATTAAT
TCTGTGGAATGTGTGTCAGTTAGGGTGTGGAAAGTCCCCAGGCTCCCCAGCAGGCAGAAGTATG
CAAAGCATGCATCTCAATTAGTCAGCAACCAGGTGTGGAAAGTCCCCAGGCTCCCCAGCAGGCA
GAAGTATGCAAAGCATGCATCTCAATTAGTCAGCAACCATAGTCCCGCCCCTAACTCCGCCCAT
CCCGCCCCTAACTCCGCCCAGTTCCGCCCATTCTCCGCCCCATGGCTGACTAATTTTTTTTATT
TATGCAGAGGCCGAGGCCGCCTCTGCCTCTGAGCTATTCCAGAAGTAGTGAGGAGGCTTTTTTG
GAGGCCTAGGCTTTTGCAAAAAGCTCCCGGGAGCTTGTATATCCATTTTCGGATCTGATCAAGA
GACAGGATGAGGATCGTTTCGCATGATTGAACAAGATGGATTGCACGCAGGTTCTCCGGCCGCT
TGGGTGGAGAGGCTATTCGGCTATGACTGGGCACAACAGACAATCGGCTGCTCTGATGCCGCCG
TGTTCCGGCTGTCAGCGCAGGGGCGCCCGGTTCTTTTTGTCAAGACCGACCTGTCCGGTGCCCT
GAATGAACTGCAGGACGAGGCAGCGCGGCTATCGTGGCTGGCCACGACGGGCGTTCCTTGCGCA
GCTGTGCTCGACGTTGTCACTGAAGCGGGAAGGGACTGGCTGCTATTGGGCGAAGTGCCGGGGC
AGGATCTCCTGTCATCTCACCTTGCTCCTGCCGAGAAAGTATCCATCATGGCTGATGCAATGCG
GCGGCTGCATACGCTTGATCCGGCTACCTGCCCATTCGACCACCAAGCGAAACATCGCATCGAG
CGAGCACGTACTCGGATGGAAGCCGGTCTTGTCGATCAGGATGATCTGGACGAAGAGCATCAGG
GGCTCGCGCCAGCCGAACTGTTCGCCAGGCTCAAGGCGCGCATGCCCGACGGCGAGGATCTCGT
CGTGACCCATGGCGATGCCTGCTTGCCGAATATCATGGTGGAAAATGGCCGCTTTTCTGGATTC
ATCGACTGTGGCCGGCTGGGTGTGGCGGACCGCTATCAGGACATAGCGTTGGCTACCCGTGATA
TTGCTGAAGAGCTTGGCGGCGAATGGGCTGACCGCTTCCTCGTGCTTTACGGTATCGCCGCTCC
CGATTCGCAGCGCATCGCCTTCTATCGCCTTCTTGACGAGTTCTTCTGAGCGGGACTCTGGGGT
TCGAAATGACCGACCAAGCGACGCCCAACCTGCCATCACGAGATTTCGATTCCACCGCCGCCTT
CTATGAAAGGTTGGGCTTCGGAATCGTTTTCCGGGACGCCGGCTGGATGATCCTCCAGCGCGGG
GATCTCATGCTGGAGTTCTTCGCCCACCCCAACTTGTTTATTGCAGCTTATAATGGTTACAAAT
AAAGCAATAGCATCACAAATTTCACAAATAAAGCATTTTTTTCACTGCATTCTAGTTGTGGTTT
GTCCAAACTCATCAATGTATCTTATCATGTCTGTATACCGTCGACCTCTAGCTAGAGCTTGGCG
TAATCATGGTCATAGCTGTTTCCTGTGTGAAATTGTTATCCGCTCACAATTCCACACAACATAC
GAGCCGGAAGCATAAAGTGTAAAGCCTGGGGTGCCTAATGAGTGAGCTAACTCACATTAATTGC
GTTGCGCTCACTGCCCGCTTTCCAGTCGGGAAACCTGTCGTGCCAGCTGCATTAATGAATCGGC
CAACGCGCGGGGAGAGGCGGTTTGCGTATTGGGCGCTCTTCCGCTTCCTCGCTCACTGACTCGC
TGCGCTCGGTCGTTCGGCTGCGGCGAGCGGTATCAGCTCACTCAAAGGCGGTAATACGGTTATC
CACAGAATCAGGGGATAACGCAGGAAAGAACATGTGAGCAAAAGGCCAGCAAAAGGCCAGGAAC
CGTAAAAAGGCCGCGTTGCTGGCGTTTTTCCATAGGCTCCGCCCCCCTGACGAGCATCACAAAA
ATCGACGCTCAAGTCAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGATACCTGTCCGCCTTT
CTCCCTTCGGGAAGCGTGGCGCTTTCTCATAGCTCACGCTGTAGGTATCTCAGTTCGGTGTAGG
TCGTTCGCTCCAAGCTGGGCTGTGTGCACGAACCCCCCGTTCAGCCCGACCGCTGCGCCTTATC
CGGTAACTATCGTCTTGAGTCCAACCCGGTAAGACACGACTTATCGCCACTGGCAGCAGCCACT
GGTAACAGGATTAGCAGAGCGAGGTATGTAGGCGGTGCTACAGAGTTCTTGAAGTGGTGGCCTA
ACTACGGCTACACTAGAAGAACAGTATTTGGTATCTGCGCTCTGCTGAAGCCAGTTACCTTCGG
AAAAAGAGTTGGTAGCTCTTGATCCGGCAAACAAACCACCGCTGGTAGCGGTTTTTTTGTTTGC
AAGCAGCAGATTACGCGCAGAAAAAAAGGATCTCAAGAAGATCCTTTGATCTTTTCTACGGGGT
CTGACGCTCAGTGGAACGAAAACTCACGTTAAGGGATTTTGGTCATGAGATTATCAAAAAGGAT
CTTCACCTAGATCCTTTTAAATTAAAAATGAAGTTTTAAATCAATCTAAAGTATATATGAGTAA
ACTTGGTCTGACAGTTACCAATGCTTAATCAGTGAGGCACCTATCTCAGCGATCTGTCTATTTC
GTTCATCCATAGTTGCCTGACTCCCCGTCGTGTAGATAACTACGATACGGGAGGGCTTACCATC
TGGCCCCAGTGCTGCAATGATACCGCGAGACCCACGCTCACCGGCTCCAGATTTATCAGCAATA
AACCAGCCAGCCGGAAGGGCCGAGCGCAGAAGTGGTCCTGCAACTTTATCCGCCTCCATCCAGT
CTATTAATTGTTGCCGGGAAGCTAGAGTAAGTAGTTCGCCAGTTAATAGTTTGCGCAACGTTGT
TGCCATTGCTACAGGCATCGTGGTGTCACGCTCGTCGTTTGGTATGGCTTCATTCAGCTCCGGT
TCCCAACGATCAAGGCGAGTTACATGATCCCCCATGTTGTGCAAAAAAGCGGTTAGCTCCTTCG
GTCCTCCGATCGTTGTCAGAAGTAAGTTGGCCGCAGTGTTATCACTCATGGTTATGGCAGCACT
GCATAATTCTCTTACTGTCATGCCATCCGTAAGATGCTTTTCTGTGACTGGTGAGTACTCAACC
AAGTCATTCTGAGAATAGTGTATGCGGCGACCGAGTTGCTCTTGCCCGGCGTCAATACGGGATA
ATACCGCGCCACATAGCAGAACTTTAAAAGTGCTCATCATTGGAAAACGTTCTTCGGGGCGAAA
ACTCTCAAGGATCTTACCGCTGTTGAGATCCAGTTCGATGTAACCCACTCGTGCACCCAACTGA
TCTTCAGCATCTTTTACTTTCACCAGCGTTTCTGGGTGAGCAAAAACAGGAAGGCAAAATGCCG
CAAAAAAGGGAATAAGGGCGACACGGAAATGTTGAATACTCATACTCTTCCTTTTTCAATATTA
TTGAAGCATTTATCAGGGTTATTGTCTCATGAGCGGATACATATTTGAATGTATTTAGAAAAAT
AAACAAATAGGGGTTCCGCGCACATTTCCCCGAAAAGTGCCACCTGACGTC
SEQ ID NO: 5-Exemplary plasmid containing E1A, E2A, E4, and VA RNA
(pLHI_Helper)
ggtacccaactccatgcttaacagtccccaggtacagcccaccctgcgtcgcaaccaggaacag
ctctacagcttcctggagcgccactcgccctacttccgcagccacagtgcgcagattaggagcg
ccacttctttttgtcacttgaaaaacatgtaaaaataatgtactaggagacactttcaataaag
gcaaatgtttttatttgtacactctcgggtgattatttaccccccacccttgccgtctgcgccg
tttaaaaatcaaaggggttctgccgcgcatcgctatgcgccactggcagggacacgttgcgata
ctggtgtttagtgctccacttaaactcaggcacaaccatccgcggcagctcggtgaagttttca
ctccacaggctgcgcaccatcaccaacgcgtttagcaggtcgggcgccgatatcttgaagtcgc
agttggggcctccgccctgcgcgcgcgagttgcgatacacagggttgcagcactggaacactat
cagcgccgggtggtgcacgctggccagcacgctcttgtcggagatcagatccgcgtccaggtcc
tccgcgttgctcagggcgaacggagtcaactttggtagctgccttcccaaaaagggtgcatgcc
caggctttgagttgcactcgcaccgtagtggcatcagaaggtgaccgtgcccggtctgggcgtt
aggatacagcgcctgcatgaaagccttgatctgcttaaaagccacctgagcctttgcgccttca
gagaagaacatgccgcaagacttgccggaaaactgattggccggacaggccgcgtcatgcacgc
agcaccttgcgtcggtgttggagatctgcaccacatttcggccccaccggttcttcacgatctt
ggccttgctagactgctccttcagcgcgcgctgcccgttttcgctcgtcacatccatttcaatc
acgtgctccttatttatcataatgctcccgtgtagacacttaagctcgccttcgatctcagcgc
agcggtgcagccacaacgcgcagcccgtgggctcgtggtgcttgtaggttacctctgcaaacga
ctgcaggtacgcctgcaggaatcgccccatcatcgtcacaaaggtcttgttgctggtgaaggtc
agctgcaacccgcggtgctcctcgtttagccaggtcttgcatacggccgccagagcttccactt
ggtcaggcagtagcttgaagtttgcctttagatcgttatccacgtggtacttgtccatcaacgc
gcgcgcagcctccatgcccttctcccacgcagacacgatcggcaggctcagcgggtttatcacc
gtgctttcactttccgcttcactggactcttccttttcctcttgcgtccgcataccccgcgcca
ctgggtcgtcttcattcagccgccgcaccgtgcgcttacctcccttgccgtgcttgattagcac
cggtgggttgctgaaacccaccatttgtagcgccacatcttctctttcttcctcgctgtccacg
atcacctctggggatggcgggcgctcgggcttgggagaggggcgcttctttttctttttggacg
caatggccaaatccgccgtcgaggtcgatggccgcgggctgggtgtgcgcggcaccagcgcatc
ttgtgacgagtcttcttcgtcctcggactcgagacgccgcctcagccgcttttttgggggcgcg
cggggaggcggcggcgacggcgacggggacgacacgtcctccatggttggtggacgtcgcgccg
caccgcgtccgcgctcgggggtggtttcgcgctgctcctcttcccgactggccatttccttctc
ctataggcagaaaaagatcatggagtcagtcgagaaggaggacagcctaaccgccccctttgag
ttcgccaccaccgcctccaccgatgccgccaacgcgcctaccaccttccccgtcgaggcacccc
cgcttgaggaggaggaagtgattatcgagcaggacccaggttttgtaagcgaagacgacgagga
tcgctcagtaccaacagaggataaaaagcaagaccaggacgacgcagaggcaaacgaggaacaa
gtcgggcggggggaccaaaggcatggcgactacctagatgtgggagacgacgtgctgttgaagc
atctgcagcgccagtgcgccattatctgcgacgcgttgcaagagcgcagcgatgtgcccctcgc
catagcggatgtcagccttgcctacgaacgccacctgttctcaccgcgcgtaccccccaaacgc
caagaaaacggcacatgcgagcccaacccgcgcctcaacttctaccccgtatttgccgtgccag
aggtgcttgccacctatcacatctttttccaaaactgcaagatacccctatcctgccgtgccaa
ccgcagccgagcggacaagcagctggccttgcggcagggcgctgtcatacctgatatcgcctcg
ctcgacgaagtgccaaaaatctttgagggtcttggacgcgacgagaaacgcgcggcaaacgctc
tgcaacaagaaaacagcgaaaatgaaagtcactgtggagtgctggtggaacttgagggtgacaa
cgcgcgcctagccgtgctgaaacgcagcatcgaggtcacccactttgcctacccggcacttaac
ctaccccccaaggttatgagcacagtcatgagcgagctgatcgtgcgccgtgcacgacccctgg
agagggatgcaaacttgcaagaacaaaccgaggagggcctacccgcagttggcgatgagcagct
ggcgcgctggcttgagacgcgcgagcctgccgacttggaggagcgacgcaagctaatgatggcc
gcagtgcttgttaccgtggagcttgagtgcatgcagcggttctttgctgacccggagatgcagc
gcaagctagaggaaacgttgcactacacctttcgccagggctacgtgcgccaggcctgcaaaat
ttccaacgtggagctctgcaacctggtctcctaccttggaattttgcacgaaaaccgcctcggg
caaaacgtgcttcattccacgctcaagggcgaggcgcgccgcgactacgtccgcgactgcgttt
acttatttctgtgctacacctggcaaacggccatgggcgtgtggcagcaatgcctggaggagcg
caacctaaaggagctgcagaagctgctaaagcaaaacttgaaggacctatggacggccttcaac
gagcgctccgtggccgcgcacctggcggacattatcttccccgaacgcctgcttaaaaccctgc
aacagggtctgccagacttcaccagtcaaagcatgttgcaaaactttaggaactttatcctaga
gcgttcaggaattctgcccgccacctgctgtgcgcttcctagcgactttgtgcccattaagtac
cgtgaatgccctccgccgctttggggtcactgctaccttctgcagctagccaactaccttgcct
accactccgacatcatggaagacgtgagcggtgacggcctactggagtgtcactgtcgctgcaa
cctatgcaccccgcaccgctccctggtctgcaattcgcaactgcttagcgaaagtcaaattatc
ggtacctttgagctgcagggtccctcgcctgacgaaaagtccgcggctccggggttgaaactca
ctccggggctgtggacgtcggcttaccttcgcaaatttgtacctgaggactaccacgcccacga
gattaggttctacgaagaccaatcccgcccgccaaatgcggagcttaccgcctgcgtcattacc
cagggccacatccttggccaattgcaagccatcaacaaagcccgccaagagtttctgctacgaa
agggacggggggtttacctggacccccagtccggcgaggagctcaacccaatccccccgccgcc
gcagccctatcagcagccgcgggcccttgcttcccaggatggcacccaaaaagaagctgcagct
gccgccgccgccacccacggacgaggaggaatactgggacagtcaggcagaggaggttttggac
gaggaggaggagatgatggaagactgggacagcctagacgaagcttccgaggccgaagaggtgt
cagacgaaacaccgtcaccctcggtcgcattcccctcgccggcgccccagaaattggcaaccgt
tcccagcatcgctacaacctccgctcctcaggcgccgccggcactgcctgttcgccgacccaac
cgtagatgggacaccactggaaccagggccggtaagtctaagcagccgccgccgttagcccaag
agcaacaacagcgccaaggctaccgctcgtggcgcgggcacaagaacgccatagttgcttgctt
gcaagactgtgggggcaacatctccttcgcccgccgctttcttctctaccatcacggcgtggcc
ttcccccgtaacatcctgcattactaccgtcatctctacagcccctactgcaccggcggcagcg
gcagcggcagcaacagcagcggtcacacagaagcaaaggcgaccggatagcaagactctgacaa
agcccaagaaatccacagcggcggcagcagcaggaggaggagcgctgcgtctggcgcccaacga
acccgtatcgacccgcgagcttagaaataggatttttcccactctgtatgctatatttcaacaa
agcaggggccaagaacaagagctgaaaataaaaaacaggtctctgcgctccctcacccgcagct
gcctgtatcacaaaagcgaagatcagcttcggcgcacgctggaagacgcggaggctctcttcag
caaatactgcgcgctgactcttaaggactagtttcgcgccctttctcaaatttaagcgcgaaaa
ctacgtcatctccagcggccacacccggcgccagcacctgtcgtcagcgccattatgagcaagg
aaattcccacgccctacatgtggagttaccagccacaaatgggacttgcggctggagctgccca
agactactcaacccgaataaactacatgagcgcgggaccccacatgatatcccgggtcaacgga
atccgcgcccaccgaaaccgaattctcctcgaacaggcggctattaccaccacacctcgtaata
accttaatccccgtagttggcccgctgccctggtgtaccaggaaagtcccgctcccaccactgt
ggtacttcccagagacgcccaggccgaagttcagatgactaactcaggggcgcagcttgcgggc
ggctttcgtcacagggtgcggtcgcccgggcgttttagggcggagtaacttgcatgtattggga
attgtagtttttttaaaatgggaagtgacgtatcgtgggaaaacggaagtgaagatttgaggaa
gttgtgggttttttggctttcgtttctgggcgtaggttcgcgtgcggttttctgggtgtttttt
gtggactttaaccgttacgtcattttttagtcctatatatactcgctctgtacttggccctttt
tacactgtgactgattgagctggtgccgtgtcgagtggtgttttttaataggtttttttactgg
taaggctgactgttatggctgccgctgtggaagcgctgtatgttgttctggagcgggagggtgc
tattttgcctaggcaggagggtttttcaggtgtttatgtgtttttctctcctattaattttgtt
atacctcctatgggggctgtaatgttgtctctacgcctgcgggtatgtattcccccgggctatt
tcggtcgctttttagcactgaccgatgttaaccaacctgatgtgtttaccgagtcttacattat
gactccggacatgaccgaggaactgtcggtggtgctttttaatcacggtgaccagtttttttac
ggtcacgccggcatggccgtagtccgtcttatgcttataagggttgtttttcctgttgtaagac
aggcttctaatgtttaaatgtttttttttttgttattttattttgtgtttaatgcaggaacccg
cagacatgtttgagagaaaaatggtgtctttttctgtggtggttccggaacttacctgccttta
tctgcatgagcatgactacgatgtgcttgcttttttgcgcgaggctttgcctgattttttgagc
agcaccttgcattttatatcgccgcccatgcaacaagcttacataggggctacgctggttagca
tagctccgagtatgcgtgtcataatcagtgtgggttcttttgtcatggttcctggcggggaagt
ggccgcgctggtccgtgcagacctgcacgattatgttcagctggccctgcgaagggacctacgg
gatcgcggtatttttgttaatgttccgcttttgaatcttatacaggtctgtgaggaacctgaat
ttttgcaatcatgattcgctgcttgaggctgaaggtggagggcgctctggagcagatttttaca
atggccggacttaatattcgggatttgcttagagacatattgataaggtggcgagatgaaaatt
atttgggcatggttgaaggtgctggaatgtttatagaggagattcaccctgaagggtttagcct
ttacgtccacttggacgtgagggcagtttgccttttggaagccattgtgcaacatcttacaaat
gccattatctgttctttggctgtagagtttgaccacgccaccggaggggagcgcgttcacttaa
tagatcttcattttgaggttttggataatcttttggaataaaaaaaaaaaaacatggttcttcc
agctcttcccgctcctcccgtgtgtgactcgcagaacgaatgtgtaggttggctgggtgtggct
tattctgcggtggtggatgttatcagggcagcggcgcatgaaggagtttacatagaacccgaag
ccagggggcgcctggatgctttgagagagtggatatactacaactactacacagagcgagctaa
gcgacgagaccggagacgcagatctgtttgtcacgcccgcacctggttttgcttcaggaaatat
gactacgtccggcgttccatttggcatgacactacgaccaacacgatctcggttgtctcggcgc
actccgtacagtagggatcgcctacctccttttgagacagagacccgcgctaccatactggagg
atcatccgctgctgcccgaatgtaacactttgacaatgcacaacgtgagttacgtgcgaggtct
tccctgcagtgtgggatttacgctgattcaggaatgggttgttccctgggatatggttctgacg
cgggaggagcttgtaatcctgaggaagtgtatgcacgtgtgcctgtgttgtgccaacattgata
tcatgacgagcatgatgatccatggttacgagtcctgggctctccactgtcattgttccagtcc
cggttccctgcagtgcatagccggcgggcaggttttggccagctggtttaggatggtggtggat
ggcgccatgtttaatcagaggtttatatggtaccgggaggtggtgaattacaacatgccaaaag
aggtaatgtttatgtccagcgtgtttatgaggggtcgccacttaatctacctgcgcttgtggta
tgatggccacgtgggttctgtggtccccgccatgagctttggatacagcgccttgcactgtggg
attttgaacaatattgtggtgctgtgctgcagttactgtgctgatttaagtgagatcagggtgc
gctgctgtgcccggaggacaaggcgtctcatgctgcgggcggtgcgaatcatcgctgaggagac
cactgccatgttgtattcctgcaggacggagcggcggcggcagcagtttattcgcgcgctgctg
cagcaccaccgccctatcctgatgcacgattatgactctacccccatgtaggcgtggacttccc
cttcgccgcccgttgagcaaccgcaagttggacagcagcctgtggctcagcagctggacagcga
catgaacttaagcgagctgcccggggagtttattaatatcactgatgagcgtttggctcgacag
gaaaccgtgtggaatataacacctaagaatatgtctgttacccatgatatgatgctttttaagg
ccagccggggagaaaggactgtgtactctgtgtgttgggagggaggtggcaggttgaatactag
ggttctgtgagtttgattaaggtacggtgatcaatataagctatgtggtggtggggctatacta
ctgaatgaaaaatgacttgaaattttctgcaattgaaaaataaacacgttgaaacataacatgc
aacaggttcacgattctttattcctgggcaatgtaggagaaggtgtaagagttggtagcaaaag
tttcagtggtgtattttccactttcccaggaccatgtaaaagacatagagtaagtgcttacctc
gctagtttctgtggattcactagaatcgatgtaggatgttgcccctcctgacgcggtaggagaa
ggggagggtgccctgcatgtctgccgctgctcttgctcttgccgctgctgaggaggggggcgca
tctgccgcagcaccggatgcatctgggaaaagcaaaaaaggggctcgtccctgtttccggagga
atttgcaagcggggtcttgcatgacggggaggcaaacccccgttcgccgcagtccggccggccc
gagactcgaaccgggggtcctgcgactcaacccttggaaaataaccctccggctacagggagcg
agccacttaatgctttcgctttccagcctaaccgcttacgccgcgcgcggccagtggccaaaaa
agctagcgcagcagccgccgcgcctggaaggaagccaaaaggagcgctcccccgttgtctgacg
tcgcacacctgggttcgacacgcgggcggtaaccgcatggatcacggcggacggccggatccgg
ggttcgaaccccggtcgtccgccatgatacccttgcgaatttatccaccagaccacggaagagt
gcccgcttacaggctctccttttgcacggtctagagcgtcaacgactgcgcacgcctcaccggc
cagagcgtcccgaccatggagcactttttgccgctgcgcaacatctggaaccgcgtccgcgact
ttccgcgcgcctccaccaccgccgccggcatcacctggatgtccaggtacatctacggattacg
tcgacgtttaaaccatatgatcagctcactcaaaggcggtaatacggttatccacagaatcagg
ggataacgcaggaaagaacatgtgagcaaaaggccagcaaaaggccaggaaccgtaaaaaggcc
gcgttgctggcgttGACGGATCGGGAGATCTCCCGATCCCCTATGGTGCACTCTCAGTACAATC
TGCTCTGATGCCGCATAGTTAAGCCAGTATCTGCTCCCTGCTTGTGTGTTGGAGGTCGCTGAGT
AGTGCGCGAGCAAAATTTAAGCTACAACAAGGCAAGGCTTGACCGACAATTGCATGAAGAATCT
GCTTAGGGTTAGGCGTTTTGCGCTGCTTCGCGATGTACGGGCCAGATATACGCGTTGACATTGA
TTATTGACTAGTTATTAATAGTAATCAATTACGGGGTCATTAGTTCATAGCCCATATATGGAGT
TCCGCGTTACATAACTTACGGTAAATGGCCCGCCTGGCTGACCGCCCAACGACCCCCGCCCATT
GACGTCAATAATGACGTATGTTCCCATAGTAACGCCAATAGGGACTTTCCATTGACGTCAATGG
GTGGAGTATTTACGGTAAACTGCCCACTTGGCAGTACATCAAGTGTATCATATGCCAAGTACGC
CCCCTATTGACGTCAATGACGGTAAATGGCCCGCCTGGCATTATGCCCAGTACATGACCTTATG
GGACTTTCCTACTTGGCAGTACATCTACGTATTAGTCATCGCTATTACCATGGTGATGCGGTTT
TGGCAGTACATCAATGGGCGTGGATAGCGGTTTGACTCACGGGGATTTCCAAGTCTCCACCCCA
TTGACGTCAATGGGAGTTTGTTTTGGCACCAAAATCAACGGGACTTTCCAAAATGTCGTAACAA
CTCCGCCCCATTGACGCAAATGGGCGGTAGGCGTGTACGGTGGGAGGTCTATATAAGCAGAGCT
CTCTGGCTAACTAGAGAACCCACTGCTTACTGGCTTATCGAAATTAATACGACTCACTATAGGG
AGACCCAAGCTGGCTAGCGTTTAAACTTAAGCTTgccaccatgagacatattatctgccacgga
ggtgttattaccgaagaaatggccgccagtcttttggaccagctgatcgaagaggtactggctg
ataatcttccacctcctagccattttgaaccacctacccttcacgaactgtatgatttagacgt
gacggcccccgaagatcccaacgaggaggcggtttcgcagatttttcccgactctgtaatgttg
gcggtgcaggaagggattgacttactcacttttccgccggcgcccggttctccggagccgcctc
acctttcccggcagcccgagcagccggagcagagagccttgggtccggtttctatgccaaacct
tgtaccggaggtgatcgatcttacctgccacgaggctggctttccacccagtgacgacgaggat
gaagagggtgaggagtttgtgttagattatgtggagcaccccgggcacggttgcaggtcttgtc
attatcaccggaggaatacgggggacccagatattatgtgttcgctttgctatatgaggacctg
tggcatgtttgtctacagtaagtgaaaattatgggcagtgggtgatagagtggtgggtttggtg
tggtaattttttttttaatttttacagttttgtggtttaaagaattttgtattgtgattttttt
aaaaggtcctgtgtctgaacctgagcctgagcccgagccagaaccggagcctgcaagacctacc
cgccgtcctaaaatggcgcctgctatcctgagacgcccgacatcacctgtgtctagagaatgca
atagtagtacggatagctgtgactccggtccttctaacacacctcctgagatacacccggtggt
cccgctgtgccccattaaaccagttgccgtgagagttggtgggcgtcgccaggctgtggaatgt
atcgaggacttgcttaacgagcctgggcaacctttggacttgagctgtaaacgccccaggccat
aaGAATTCTGCAGATATCCAGCACAGTGGCGGCCGCTCGAGTCTAGAGGGCCCGTTTAAACCCG
CTGATCAGCCTCGACTGTGCCTTCTAGTTGCCAGCCATCTGTTGTTTGCCCCTCCCCCGTGCCT
TCCTTGACCCTGGAAGGTGCCACTCCCACTGTCCTTTCCTAATAAAATGAGGAAATTGCATCGC
ATTGTCTGAGTAGGTGTCATTCTATTCTGGGGGGTGGGGTGGGGCAGGACAGCAAGGGGGAGGA
TTGGGAAGACAATAGCAGGCATGCTGGGGATGCGGTGGGCTCTATGGCTTCTGAGGCGGAAAGA
ACCAGCTGGGGCTCTAGGGGGTATCCCCtttccataggctccgcccccctgacgagcatcacaa
aaatcgacgctcaagtcagaggtggcgaaacccgacaggactataaagataccaggcgtttccc
cctggaagctccctcgtgcgctctcctgttccgaccctgccgcttaccggatacctgtccgcct
ttctcccttcgggaagcgtggcgctttctcatagctcacgctgtaggtatctcagttcggtgta
ggtcgttcgctccaagctgggctgtgtgcacgaaccccccgttcagcccgaccgctgcgcctta
tccggtaactatcgtcttgagtccaacccggtaagacacgacttatcgccactggcagcagcca
ctggtaacaggattagcagagcgaggtatgtaggcggtgctacagagttcttgaagtggtggcc
taactacggctacactagaagaacagtatttggtatctgcgctctgctgaagccagttaccttc
ggaaaaagagttggtagctcttgatccggcaaacaaaccaccgctggtagcggtggtttttttg
tttgcaagcagcagattacgcgcagaaaaaaaggatctcaagaagatcctttgatcttttctac
ggggtctgacgctcagtggaacgaaaactcacgttaagggattttggtcatgagattatcaaaa
aggatcttcacctagatccttttaaattaaaaatgaagttttaaatcaatctaaagtatatatg
agtaaacttggtctgacagTTAGAAAAACTCATCGAGCATCAAATGAAACTGCAATTTATTCAT
ATCAGGATTATCAATACCATATTTTTGAAAAAGCCGTTTCTGTAATGAAGGAGAAAACTCACCG
AGGCAGTTCCATAGGATGGCAAGATCCTGGTATCGGTCTGCGATTCCGACTCGTCCAACATCAA
TACAACCTATTAATTTCCCCTCGTCAAAAATAAGGTTATCAAGTGAGAAATCACCATGAGTGAC
GACTGAATCCGGTGAGAATGGCAAAAGTTTATGCATTTCTTTCCAGACTTGTTCAACAGGCCAG
CCATTACGCTCGTCATCAAAATCACTCGCATCAACCAAACCGTTATTCATTCGTGATTGCGCCT
GAGCGAGACGAAATACGCGATCGCTGTTAAAAGGACAATTACAAACAGGAATCGAATGCAACCG
GCGCAGGAACACTGCCAGCGCATCAACAATATTTTCACCTGAATCAGGATATTCTTCTAATACC
TGGAATGCTGTTTTTCCGGGGATCGCAGTGGTGAGTAACCATGCATCATCAGGAGTACGGATAA
AATGCTTGATGGTCGGAAGAGGCATAAATTCCGTCAGCCAGTTTAGTCTGACCATCTCATCTGT
AACATCATTGGCAACGCTACCTTTGCCATGTTTCAGAAACAACTCTGGCGCATCGGGCTTCCCA
TACAAGCGATAGATTGTCGCACCTGATTGCCCGACATTATCGCGAGCCCATTTATACCCATATA
AATCAGCATCCATGTTGGAATTTAATCGCGGCCTCGACGTTTCCCGTTGAATATGGCTCATact
cttcctttttcaatattattgaagcatttatcagggttattgtctcatgagcggatacatattt
gaatgtatttagaaaaataaacaaataggggttccgcgcacatttccccgaaaagtgccaccta
aattgtaagcgttaatattttgttaaaattcgcgttaaatttttgttaaatcagctcatttttt
aaccaataggccgaaatcggcaaaatcccttataaatcaaaagaatagaccgagatagggttga
gtgttgttccagtttggaacaagagtccactattaaagaacgtggactccaacgtcaaagggcg
aaaaaccgtctatcagggcgatggcccactacgtgaaccatcaccctaatcaagttttttgggg
tcgaggtgccgtaaagcactaaatcggaaccctaaagggagcccccgatttagagcttgacggg
gaaagccggcgaacgtggcgagaaaggaagggaagaaagcgaaaggagcgggcgctagggcgct
ggcaagtgtagcggtcacgctgcgcgtaaccaccacacccgccgcgcttaatgcgccgctacag
ggcgcgatggatcc

1. A method of producing an adeno-associated virus (AAV) in an E1 complementary producer cell, comprising:
a. transfecting the E1 complementary producer cell with one or more vectors comprising:
i. an E1A adenovirus helper gene;
ii. an adenovirus helper gene selected from E2A, E4, or both;
iii. a viral-associated, non-coding RNA (VA RNA); and
iv. an AAV gene selected from Rep, Cap, or both;
b. culturing the transfected E1 complementary producer cell under conditions suitable for producing the AAV; and
c. purifying the AAV from the cultured E1 complementary producer cell, thereby obtaining the AAV.

2. The method of claim 1, wherein (i), (ii), (iii), and (iv) are on a single vector, or wherein (i), (ii), (iii), and (iv) are on more than one vector.

3. (canceled)

4. The method of claim 2, wherein (i), (ii), and (iii) are on a first vector, and (iv) is a second vector;
or wherein (i) is on a first vector, (ii) and (iii) are on a second vector, and (iv) is on a third vector;
or wherein each of (i), (ii), (iii), and (iv) is on a separate vector.

5. (canceled)

6. (canceled)

7. The method of claim 4, wherein (i) is on a separate vector from (ii), (iii), and (iv), and wherein the vector comprising (i) is transfected into the E1 complementary producer cell at a ratio of about 1:1 to about 5:1 to each of the vectors comprising (ii), (iii), and (iv).

8. The method of claim 7, wherein the ratio of the vector comprising (i) to each of the vectors comprising (ii), (iii), and (iv) transfected into the E1 complementary producer cell is 1:1, 2:1, or 3:1.

9. (canceled)

10. (canceled)

11. The method of claim 1, wherein (i), (ii), (iii), and (iv) are operably linked to one or more promoters.

12. (canceled)

13. The method of claim 12, wherein each of (i), (ii), (iii), and (iv) is operably linked to a separate promoter, and wherein the promoter operably linked to (i) provides an expression level that is substantially the same, or at least 2-fold higher, or at least 3-fold higher as compared to the promoters operably linked to (ii), (iii), and (iv).

14. (canceled)

15. (canceled)

16. The method of claim 11, wherein (i) is operably linked to a promoter and further operably linked to an enhancer, and wherein (ii), (iii), and (iv) are not operably linked to an enhancer.

17. The method of claim 1, wherein a copy number ratio of (i) to each of (ii), (iii), and (iv) on the one or more vectors is about 1:1 to about 5:1.

18. (canceled)

19. (canceled)

20. (canceled)

21. The method claim 1, wherein (ii) comprises E2A and E4; and/or wherein (iv) comprises Rep and Cap.

22. (canceled)

23. The method of claim 1, wherein the culturing comprises expressing E1A at a ratio of about 1:1 to about 5:1 to each of (ii), (iii), and (iv).

24. (canceled)

25. The method of claim 1, wherein the one or more vectors further comprises:
(v) one or both of:
(A) a helper gene selected from E1B, NP1, NS2, or a combination thereof, optionally wherein the E1B is E1B-19k or E1B-55k; and
(B) a gene of interest.

26. (canceled)

27. (canceled)

28. The method of claim 25, wherein (i) and (v) are on a single vector, or wherein (v) is on a separate vector from (i), (ii), (iii), and (iv).

29. (canceled)

30. (canceled)

31. (canceled)

32. The method of claim 1, wherein the method produces at least 1.5-fold or higher titer of the AAV as compared to a method that does not comprise transfecting a E1 complementary producer cell with an E1A adenovirus helper gene.

33. A method of producing an adeno-associated virus (AAV) in an E1 complementary producer cell, comprising:
a. transfecting the E1 complementary producer cell with:
i. a first vector comprising E1A operably linked to a first promoter;
ii. a second vector comprising E2A, E4, and a viral-associated, non-coding RNA (VA RNA), all operably linked to a second promoter;
iii. a third vector comprising Rep and Cap, both operably linked to a third promoter; and
iv. optionally, one or both of:
(A) a helper gene selected from E1B, NP1, NS2, or a combination thereof, wherein the helper gene is on the first, second, or third vector, or wherein the helper gene is on a fourth vector; and
(B) a gene of interest, wherein the gene of interest is on the first, second, third, or fourth vector, or wherein the gene of interest is on a fifth vector,
wherein a transfection ratio of (i) to each of (ii) and (iii) is about 1:1 to about 3:1;
b. culturing the transfected E1 complementary producer cell under conditions suitable for producing the AAV; and
c. purifying the AAV from the cultured E1 complementary producer cell, thereby obtaining the AAV.

34. A method of producing an adeno-associated virus (AAV) in an E1 complementary producer cell, comprising:
a. transfecting the E1 complementary producer cell with:
i. a first vector comprising E1A operably linked to a first promoter and an enhancer;
ii. a second vector comprising E2A, E4, and a viral-associated, non-coding RNA (VA RNA), all operably linked to a second promoter;
iii. a third vector comprising Rep and Cap, each operably linked to a third promoter; and
iv. optionally, one or both of:
(A) a helper gene selected from E1B, NP1, NS2, or a combination thereof, wherein the helper gene is on the first, second, or third vector, or wherein the helper gene is on a fourth vector; and
(B) a gene of interest, wherein the gene of interest is on the first, second, third, or fourth vector, or wherein the gene of interest is on a fifth vector:
b. culturing the transfected E1 complementary producer cell under conditions suitable for producing the AAV, wherein E1A is expressed at a ratio of about 1:1 to about 3:1 to each of E2A, E4, VA RNA, Rep, and Cap; and
c. purifying the AAV from the cultured E1 complementary producer cell, thereby obtaining the AAV.

35. A method of producing an adeno-associated virus (AAV) in an E1 complementary producer cell, comprising:
a. transfecting the E1 complementary producer cell with:
i. a first vector comprising E1A, E2A, E4 and VA RNA, all operably linked to a first promoter;
ii. a second vector comprising Rep and Cap, operably linked to a second promoter; and
iii. optionally, one or both of:
(A) a helper gene selected from E1B, NP1, NS2, or a combination thereof, wherein the helper gene is on the first or second vector, or wherein the helper gene is on a third vector; and
(B) a gene of interest, wherein the gene of interest is on the first, second, or third vector, or wherein the gene of interest is on a fourth vector,
wherein a copy number ratio of E1A to each of E2A, E4, VA RNA, Rep, and Cap is about 1:1 to about 3:1;
b. culturing the transfected E1 complementary producer cell under conditions suitable for producing the AAV; and
c. purifying the AAV from the cultured E1 complementary producer cell, thereby obtaining the AAV.

36. (canceled)

37. (canceled)

38. (canceled)

39. (canceled)

40. (canceled)

41. (canceled)

42. The method of claim 1, wherein the E1 complementary producer cell is a HEK293 cell, a 911 cell, a pTG6559 cell, a PER.C6 cell, a GH329 cell, an N52.E6 (CAP®) cell, a HeLa-E1 cell, an UR cell, a VLI-293 cell, an Ac51 cell, an Ac139 cell, or a variant or derivative thereof.

43. The method of claim 42, wherein the E1 complementary producer cell is a HEK293 cell, a PER.C6 cell, or a N52.E6 (CAP®) cell.

44. (canceled)

45. The method of claim 43, wherein the E1 complementary producer cell is a HEK293 cell, and wherein the HEK293 cell is a HEK293S cell, a HEK293T cell, a HEK293F cell, a HEK293FT cell, a HEK293FTM cell, a HEK293SG cell, a HEK293SGGD cell, a HEK293H cell, a HEK293E cell, a HEK293MSR cell, a HEK293A cell, or a combination thereof.

46. (canceled)

47. (canceled)

48. (canceled)
